SEQUENCE LISTING (12 sequences)

SEQ ID NO: 1 - DNA, 18 bp, Artificial Sequence
  Description of Artificial Sequence primer
caaggtccccgtttctcc

SEQ ID NO: 2 - DNA, 18 bp, Artificial Sequence
  Description of Artificial Sequence primer
caggggatagtggtgtgg

SEQ ID NO: 3 - DNA, 17 bp, Artificial Sequence
  Description of Artificial Sequence primer
ccccaccacagaatcca

SEQ ID NO: 4 - DNA, 18 bp, Artificial Sequence
  Description of Artificial Sequence primer
gctgccgtccttgtctct

SEQ ID NO: 5 - DNA, 17 bp, Artificial Sequence
  Description of Artificial Sequence primer
gcccccctgatctttcc

SEQ ID NO: 6 - DNA, 17 bp, Artificial Sequence
  Description of Artificial Sequence primer
ctggggcggtcaataat

SEQ ID NO: 7 - DNA, 18 bp, Artificial Sequence
  Description of Artificial Sequence primer
gatgtggaatgtgtgcga

SEQ ID NO: 8 - DNA, 16 bp, Artificial Sequence
  Description of Artificial Sequence primer
cgccaggagtttgaca

SEQ ID NO: 9 - DNA, 18 bp, Artificial Sequence
  Description of Artificial Sequence primer
ctcattcctcccactcat

SEQ ID NO: 10 - DNA, 17 bp, Artificial Sequence
  Description of Artificial SequencePCR primer
ccccaccacagaatcca

SEQ ID NO: 11 - DNA, 18 bp, Artificial Sequence
  Description of Artificial SequencePCR Primer
caaggtccccgtttctcc

SEQ ID NO: 12 - DNA, 17 bp, Artificial Sequence
  Description of Artificial SequencePCR primer
gcccccctgatctttcc

(19) United States
(12) Patent Application Publication
(10) Pub. No.: US 2006/0168673 A1
(43) Pub. Date: Jul. 27, 2006

(54) Transgenic animals as models for neurodegenerative disease

(76) Inventors: Hugo A.G. Geerts, Beerse (BE); Koenraad F.F. Spittaels, Puurs (BE); Chris Van de Haute, Leuven (BE); Freddy K. Van Leuven, Linden (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/243,412

(22) Filed: Oct. 4, 2005

Related U.S. Application Data
(63) Continuation of application No. 10/030,308, filed on Jan. 2, 2002.

(30) Foreign Application Priority Data
Jul. 2, 1999 (GB) 9915576.9
Feb. 4, 2000 (GB) 0002674.0

(51) Int. Cl. A01K 67/027; C07H 21/04; C12N 15/87

(52) U.S. Cl. 800/14; 800/21; 800/18; 435/455; 536/23.2

(57) ABSTRACT

The present invention relates to cell and animal models for a disease condition and in particular to an animal model which can function as a model for neurodegenerative diseases, such as Alzheimers.

### FIELD OF THE INVENTION

The present invention relates to cell and animal models for a disease condition and in particular to an animal model which can function as a model for neurodegenerative diseases, such as Alzheimers.

### BACKGROUND OF THE INVENTION

Alzheimers disease is a neurodegenerative disorder which is the most prevalent form of senile dementia, with approximately 5% of individuals of 65 and 20% of those over so being afflicted. The disease is characterised by the appearance of two principal lesions within the brain termed neurofibrillary tangles and senile plaques.

Neurofibrillary tangles are intracellular inclusion bodies which comprise filamentous aggregates of paired helical filaments (PHF). The principal component of PHF has been shown to be Tau, a microtubule associated protein involved in stabilising the cytoskeleton and in determining neuronal shape. Tau is a phosphoprotein and aberrant hyper phosphorylation of Tau appears to represent one mechanism for its aggregation into PHF. Biochemical analysis and structural prediction of the phosphorylation sites of human protein Tau of paired helical filaments (PHF) in brain of Alzheimer's disease (AD) patients revealed that many sites consist of serine or threonine residues followed by a proline residue, focussing attention on proline dependent kinases (Wood et al., 1986; Wischik et al., 1988; Brion et al., 1991; Hasegawa et al., 1992; Pollanen et al., 1997).

Further neurodegenerative disorders mediated by Tau positive filamentous lesions include, FTDP-17 (Fronto-temporal dementia associated with Parkinson=s disease), Cortico-basal degeneration, progressive supranuclear palsy, multiple system atrophy , Pick=s disease, Dementia Pugilistica, Dementia with tangles only, dementia with tangles and calcification, Down syndrome, Myotonic dystrophy, Niemann Pick=s disease type C, Parkinsonism-dementia complex of Guam, Postencephalic Parkinsonism, Prion diseases with tangles, subacute sclerosing panencephalitis.

Despite the data currently available, convincing evidence in addition to a suitable animal model demonstrating and exhibiting the phosphorylation of protein Tau by human kinases in vivo, is lacking.

### SUMMARY OF THE INVENTION

The present invention is therefore directed to providing an animal model of neurodegenerative diseases, such as Alzheimers and which model may be utilised to identify compounds useful in treating or ameliorating the symptoms of the condition.

In a first aspect the present invention provides a nucleic acid vector comprising a) a nucleic acid sequence encoding a human Tau protein; b) a sequence capable of directing expression of said Tau protein in the nervous system of said animal; and c) a sequence which facilitates integration of said vector into the genome of said animal so as to prevent functional expression of said animal Tau protein in favour of said human Tau protein. This construct or vector thus permits generation of cells of non human animals which express the human Tau and which are substantially uncontaminated with endogenous Tau proteins from the animal or cell. Thus, such a cell or non-human animal may be particularly useful as a model to monitor the function of human Tau proteins and its potential role in the progression of neurodegenerative disorders mediated by Tau protein, such as Alzheimer's disease.

### BRIEF DESCRIPTION OF THE FIGURES

FIG. 1: is an illustration of the recombinant DNA construct used to target the mouse Tau locus. The triangles represent the loxP sites. The black boxes indicate a part of the exon 1 of the mouse Tau gene. BSSK+ denotes the bluescript cloning vector. Pgk-hyg represents the hygromycin marker gene. The middle figure shows a partial structure of the wild-type mouse Tau gene. Nco 1 is the unique site on exon 1 into which the entire construct is introduced. The lower figure shows the construct ready for introduction into the ES cells and if homologous recombination occurs in the mouse genome, the different probes used with different enzyme digestions. Details are in the text under the section.

FIG. 2: is an illustration of the Southern Blot used to identify transgenic mice incorporating the human Tau 40 cDNA at the embryonic stage. 5 of the 46 pups injected at the embryonic stage contained the DNA.

FIG. 3: is an illustration of a Western Blot results indicating a 64 kDa Tau protein in three different transgenic mouse strains, and probed with antibodies HT-7 and Tau-5.

FIGS. 4 & 5: are illustrations of the different digestions using rare cutting restriction enzymes in a restriction map of the human Tau gene.

FIG. 6: is an illustration of the expression of human GSK-3β in brain of transgenic mice (A) and activity of human GSK-3β in the brain of transgenic animals using a synthetic substrate peptide (B).

FIG. 7: is an illustration of the results of a Western Blot of brain extracts of GSK-3β[S9A]/htau40 double transgenic mice, 5 weeks old. Brain extracts from wild-type (WT), GSK-3β[S9A] single transgenic ([S9A]-5), htau40 single transgenic and GSK-3β[S9A]/htau40 double transgenic mice were immunoblotted with the specified monoclonal antibodies. For Tau-5 immunodetection, 6 times less extract was applied than for AT8 and AT-180 staining. Intense hyperphosphorylation of human protein tau was evident by reaction with monoclonal antibodies AT-8 and AT-180 in the double transgenic animals of all three lines generated. Relative Mr is indicated on the left in kDa. The single and double accolades on the right denote the endogenous murine and the transgenic human protein tau respectively.

FIG. 8: is an illustration of the method of producing the loxP—hygromycin construct. This construct is incapable of replication and/or of expressing exogenous proteins in yeast.

FIG. 9: is a restriction digest of the construct of FIG. 8 using various restriction enzymes.

FIG. 10: illustrates a restriction map of the construct of FIG. 8.

FIG. 11: is an illustration of the results obtained by probing a cell line to ensure the presence of the constructs.

FIG. 12: is an illustration of Western Blotting of brain extracts of GSK-3β[S9A] transgenic mice of 7 months old. Each panel compares brain extracts from 2 individual wild type (wt) mice and from 2 individual GSK-3β[S9A] transgenic mice, all about 7 months old, immunoblotted with antibodies Tau-5, PHF-1, AT-8 and AT180 as indicated with each panel. Brain homogenates were purified from mouse IgG prior to electrophoresis.

FIG. 13: is an illustration of the effect of alkaline phosphatase pretreatment on hyperhosphoryled protein tau. Brain homogenates of single and double htau-40-5 and GSK-3β[S9A]/htau40 transgenic mice were either applied untreated, or after incubation at 37° C. for 3 hours without or with alkaline phosphatase (0.5 unites per Fl) prior to Wester Blotting. For staining with antibodies Tau-5 and Tau-1, the amounts of extract applied were 6 times less than for blotting with AT-8 and AT-180. Note the reduction in signals and the increase in electrophoretic mobility as described and discussed in the text.

FIG. 14: (a) and (e) are graphic representations of the recombinant DNA constructs used to generate transgenic mice that express a mutant form of GSK-3β, denoted GSK3-β[S9A] and htau40; (b) and (c) are illustrations of the results obtained from a Western Blot of brain and spinal cord extracts from transgenic and wild type mice, illustrating expression of the transgene in the transgenic compared to the wild-type mice; (d) and (g) are illustrations of immunohistochemical localisation of the transgenic proteins in neuronal cell bodies and processes in the cortex and hippocampus in addition to motor neurons in the ventral horn of the spinal cord, expressing both the human GSK-3β[S9A] mutant and the human tau transgene.

FIG. 15: is an illustration of the results obtained from a Western Blot using brain extracts of double tau-4R×GSK-3β[S9A] transgenic mice of five weeks old immunoblotted using antibodies, AT-8, AT-180, AD-2 and 12E8.

FIG. 16: is an illustration of the results obtained from binding experiments of tau protein to re-assembled microtubules extracted from mouse brain and spinal cord derived from htau-4R×GSK-3β double transgenic mice compared with htau-4R littermates.

FIG. 17: is an illustration of results obtained from Western Blots of human and murine tau protein which remained unbound to microtubules, using antibodies Tau-1, AT-180 and AD-2. Further shown are the results of quantitative analysis of the unbound protein by densitometric scanning and normalisation to the reaction with antibody Tau-5.

FIG. 18: is an illustration of the results obtained from a Western Blot to demonstrate that AD-2 and 12E8 epitopes are differentially present on the bound and free protein tau in the microtubule extracts.

FIG. 19: is an illustration of sections of diseased axons showing accumulation of synapthophysin-bearing vesicles in human tau transgenic animals.

FIG. 20: is an illustration of sections of brain and spinal cord of double tau-4R×GSK-3β transgenic mice showing a dramatic reduction in the number of dilated axons and lack of muscle wasting in the quadriceps of htau 40-1×GSK3β mice.

FIG. 21: is an illustration of the results obtained from evaluating the effect of co-expression of GSK-3β on the motoric aspect of the phenotype in different tests in double htau 40×GSK-3β transgenic mice, relative to htau 40-2, GSK-3β and Wild-type mice. (a) is the result of the >uprighting reflex=, (b) a rodwalking test measuring the number of mice that dropped off the rod and (c) the time the mice remained on the rod (d) the forced swim test and (e) the grid hang test.

### DETAILED DESCRIPTION OF THE PREFERRED EMBODIMENTS

In one embodiment of the invention, the sequence which facilitates integration of the vector into the genome comprises a sequence of nucleotides which exhibits a sufficient degree of homology with the Tau sequence of the animal or the flanking regions thereof, to permit homologous recombination and subsequent insertion of the vector into the genome of said animal at a location which disrupts the coding region and hence expression of the endogenous Tau in said animal in favour of the human Tau protein encoded from the sequence present on said vector. Whilst it will be appreciated by the skilled practitioner that a range of sites upstream, downstream or within the endogenous Tau sequence in the animal genome may be utilised as the site of homologous recombination, it is preferred that the region of homology is selected such that expression of proteins from other gene coding sequences upstream or downstream of the endogenous Tau sequence are not affected. As discussed in more detail in the example below, the vector of the invention may be targeted to, for example, the corresponding Tau sequence of a mouse by the inclusion of a NcoI restriction fragment suitable for insertion of the vector into the unique NcoI site in exon 1 of the Tau sequence in the mouse genome, although as aforementioned a range of appropriate regions of homology to sites upstream or downstream of said Tau sequence may be used.

An expression vector according to the invention includes a vector having a nucleic acid according to the invention operably linked to regulatory sequences, such as promoter regions, that are capable of effecting expression of said DNA fragments. The term “operably linked” refers to a juxta position wherein the components described are in a relationship permitting them to function in their intended manner. Such vectors may be transformed into a suitable host cell to provide for expression of a polypeptide according to the invention. Thus, in a further aspect, the invention provides a process for preparing receptors according to the invention which comprises cultivating a host cell, transformed or transfected with an expression vector as described above under conditions to provide for expression by the vector of a coding sequence encoding the receptors, and recovering the expressed receptors.

The vector according to the invention is termed a “knock in-knock out” vector by virtue of the fact that the endogenous Tau protein is prevented from being expressed in favour of the exogenous DNA sequence. Preferably, such a vector further comprises a marker sequence which in one embodiment may comprise the hygromycin marker gene Pgk-hyg.

The sequence encoding the Tau protein is preferably a cDNA sequence, and even more preferably encodes one of the Tau 40 isoforms already known in the art (Goedert M, Trends Neuroscience 1993 November; 16(11): 460-465). However, although the known sequences encoding human Tau isoforms may be utilised, mutated Tau sequences may be used to investigate the role of Tau protein in the pathology of neurodegenerative disorders in an animal mediated by Tau protein.

A second aspect of the invention comprises a further nucleic acid vector comprising (a) a nucleic acid sequence encoding a protein capable of modulating a human Tau protein; (b) a sequence capable of directing expression of said protein in the cells of said animal; and (c) a targeting sequence capable of facilitating integration of said vector into the genome of said animal optionally at a position corresponding to a sequence in said animal equivalent to said protein capable of modulating human Tau protein, so as to prevent expression of said equivalent sequence in favour of said protein capable of modulating human Tau protein.

Such a vector when integrated at said equivalent sequence in the animal genome, in a similar fashion to the vector described above, permits expression of the protein capable of modulating Tau protein in favour of the related or equivalent protein in said animal.

The sequence capable of directing expression of said human Tau protein or the modulator thereof is preferably a transcriptional control sequence which can steer expression of the proteins to the nervous system of the non-human animal. Transcriptional control sequences according to the invention comprise a suitable promoter and other regulatory regions, such as enhancer sequences, that can modulate the activity of the promoter.

Regulatory elements required for expression include promoter sequences to bind RNA polymerase and transcription initiation sequences for ribosome binding. For example, a bacterial expression vector may include a promoter such as the lac promoter and for transcription initiation in the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or assembled from the sequences described by methods well known in the art.

A promoter refers to the region of DNA that is upstream with respect to the direction of transcription of the transcription initiation site and which promoter is in a relationship permitting expression of the relevant proteins according to the invention.

DNA sequences that drive expression to neurons are known. They include both control systems that are neuron-specific and control systems that are more or less promiscuous but that induce high levels of expression in neurons. Depending on the nature of the construct used in the production of the transgenic animal and, in particular, the control elements, the desired proteins may be expressed in all neurons or only in restricted subsets of neurons of transgenic animals. Neuron-specific control systems, that drive expression to neuronal cell types in general, are known. They may be derived from genes encoding neuron-specific proteins. Such systems may be used to bring about expression of the desired Tau protein and/or the protein capable of it's modulation, in neurons.

Preferably the sequence is a promoter which directs expression of said proteins in the neurons of the brain or other such cells including astrocytes, oligodendrocytes microglia or Schwann cells. Preferably, the promoter is the mouse Thy-1 promoter which drives expression in mouse central neurons.

The vector according to this aspect of the invention may, advantageously, be used in combination with the vector incorporating the sequence encoding the human Tau protein described above to transfect a non-human animal and thus provide a transgenic animal which serves as a model permitting investigation into the interactions between the protein capable of modulating human Tau protein and said Tau protein and identification of potential therapeutic agents capable of modulating the effects of the phosphorylation of Tau.

Alternatively, a transgenic animal incorporating the nucleic acid vector encoding said human Tau protein may be crossed with another transgenic animal comprising the vector encoding said protein capable of modulating human Tau protein which may result in offspring which express both of the proteins.

The human sequences introduced into the transgenic animals may encode those which are known in the art. Preferably, the human Tau comprises a human Tau isoform already known in the art. Alternatively, the sequence may have been subject to a mutation, such as for example, a point mutation which may simulate a mutation that gives rise to certain genetic diseases. There are techniques known in the art to mutate a desired genetic region so as to inactivate or alter function or expression of the protein. Such techniques include homologous recombination. Methods for detecting homologous recombinant events include the polymerase chain reaction or by using marker or reporter genes which are only expressed in the event of a successful targeted recombinant event. Such mutated sequences when expressed in a transgenic non-human animal can advantageously, be used to investigate their effect on the phenotype of said animal and its role in the progression of neurodegenerative disease, such as Alzheimers mediated by Tau protein.

In a preferred embodiment of the second aspect of the invention, the protein capable of modulating human Tau protein is a kinase, and preferably one which is capable of phosphorylating human Tau protein, such as human GSK-3β kinase, for example. In vitro assays have identified glycogen synthase kinase-3β (GSK-3β) as one candidate involved in phosphorylation of Tau. Phosphorylation by GSK-3β of bovine (Ishiguro et al., 1992a and 1992b) and human protein Tau (Hanger et al., 1992; Mandelkow et al., 1992) in cell-free systems, resulted in phosphorylation patterns of protein Tau that resembled those of the protein isolated from PHF from AD brain (Ishiguro et al., 1993). The in vitro phosphorylation of human recombinant protein Tau by GSK-3β reduced its ability to induce microtubule nucleation (Utton et al., 1997), while the kinase also phosphorylated neurofilament proteins on specific domains (Guan et al., 1991). Further evidence for GSK-3β as a potential protein Tau and neurofilament kinase has been obtained in transfected cells, wherein both protein Tau (Lovestone et al., 1994; Anderton et al., 1995; Lovestone et al., 1996; Lovestone and Reynolds, 1997) and NF—H were identified as substrates. Co-transfection of GSK-3β with Tau in CHO cells increased its phosphorylation concomitant with loss of prominent bundles of microtubules (Wagner et al., 1996), while co-transfection with NF—H in COS cells caused electrophoretic mobility retardation and the appearance of phosphate-dependent antibody profiles.

The involvement of GSK-3β in the hyperphosphorylation of Tau, both in cultured neurons and in vivo in brain, was indirectly supported by the finding that lithium, as inhibitor of GSK-3β, caused Tau dephosphorylation at the sites recognized by antibodies Tau-1 and PHF-1, which are two of the major epitopes typically associated with PHF in AD brain. The physiological role of GSK-3β was proposed to be in stabilizing the neuronal cytoskeleton by controlling phosphorylation of Tau and neurofilament-H and eventually other substrates (Takahashi et al., 1994). In addition, GSK-3β plays a role in the development of the brain of Xenopus as part of the Wingless signaling pathway in which the kinase is a negative regulator of dorsoventral axis formation. In this mechanism, phosphorylation of β-catenin, mediated by axin or conductin, controls the degradation of β-catenin by the ubiquitin-proteasome pathway (Aberle et al., 1997; Behrens et al. 1998; Ikeda et al., 1998). Thus, the model is particularly useful to investigate the molecular basis for Alzheimers and other neurodegenerative disorders mediated by Tau protein and to investigate compounds which may alleviate the symptoms of the disease.

The vectors may be transformed into a suitable host cell which is preferably eukaryotic, which may itself be used to transform a non-human animal. Thus, in a further aspect the invention provides a process for preparing human Tau protein or a protein capable of modulating Tau protein, comprising cultivating a host cell transformed or transfected with a vector according to the invention, under conditions to provide for expression by the vector of said proteins, and recovering the expressed proteins. Preferably, the host cell is a non-human animal cell, and even more preferably, an embryonic cell of a non-human animal.

Incorporation of the nucleic acid sequences into the vector according to the invention for subsequent transformation and integration into the genome of said host cell or non-human animal is carried out by procedures well known to those skilled in the art as provided in Sambrook et al., Molecular Cloning, A Laboratory Manual, Cold Spring Harbour Laboratory Press. The vector may be introduced by transfection or other suitable techniques such as electroporation. In the present invention, the incorporation of the exogenous DNA into the genome of the animal is accomplished by electroporation of the vector in embryonic stem cells. The cells that have the exogenous DNA incorporated into their genome by homologous recombination may subsequently be injected into blastocysts for generation of the transgenic animals with the desired phenotype. Successfully transformed cells which contain the vector according to the invention may be identified by well known techniques, such as lysing the cells and examining the DNA by, for example, Southern blotting or using the polymerase chain reaction.

The vectors may be, for example, plasmid, virus, cosmid or phage vectors, and may contain one or more selectable markers such as the hygromycin marker gene Pgk-hyg.

The present invention also advantageously provides nucleic acid sequences of at least approximately 10 contiguous nucleotides of a nucleic acid according to the invention and preferably from 10 to 50 nucleotides even more preferably, the nucleic acid sequence comprise the sequences illustrated in Table 1. These sequences may, advantageously be used as probes or primers to initiate replication, or the like. Such nucleic acid sequences may be produced according to techniques well known in the art, such as by recombinant or synthetic means. They may also be used in diagnostic kits or the like for detecting the presence of a nucleic acid according to the invention. These tests generally comprise contacting the probe with the sample under hybridising conditions and detecting for the presence of any duplex or triplex formation between the probe and any nucleic acid in the sample.

The probes according to this aspect of the invention may be anchored to a solid support. Preferably, they are present on an array so that multiple probes can simultaneously hybridize to a single biological sample. The probes can be spotted onto the array or synthesised in situ on the array. (See Lockhart et al., Nature Biotechnology, vol. 14, December 1996 “Expression monitoring by hybridisation to high density oligonucleotide arrays”. A single array can contain more than 100, 500 or even 1,000 different probes in discrete locations.

The nucleic acid sequences, according to the invention may be produced using such recombinant or synthetic means, such as for example using PCR cloning mechanisms which generally involve making a pair of primers, which may be from approximately 10 to 50 nucleotides to a region of the gene which is desired to be cloned, bringing the primers into contact with mRNA, cDNA, or genomic DNA from a human cell, performing a polymerase chain reaction under conditions which bring about amplification of the desired region, isolating the amplified region or fragment and recovering the amplified DNA. Generally, such techniques are well known in the art, such as described in Sambrook et al. (Molecular Cloning: a Laboratory Manual, 1989).

The nucleic acids or oligonucleotides according to the invention may carry a revealing label. Suitable labels include radioisotopes such as ³²P or ³⁵S, enzyme labels or other protein labels such as biotin or fluorescent markers. Such labels may be added to the nucleic acids or oligonucleotides of the invention and may be detected using known techniques per se.

Antisense technology can be used to control gene expression through triple-helix formation of antisense DNA or RNA, both of which methods are based on binding of a polynucleotide to DNA or RNA. For example, the 5′ coding portion or the mature protein sequence, which encodes for the protein of the present invention, is used to design an antisense RNA oligonucleotide of from 10 to 50 base pairs in length. A DNA oligonucleotide is designed to be complementary to a region of the gene involved in transcription (triple-helix—see Lee et al. Nucl. Acids Res., 6:3073 (1979); Cooney et al., Science, 241:456 (1988); and Dervan et al., Science, 251: 1360 (1991), thereby preventing transcription and the production of human Tau or the protein capable of modulating Tau according to the invention defined herein. The antisense RNA oligonucleotide hybridises to the mRNA in vivo and blocks translation of an mRNA molecule (antisense—Okano, J. Neurochem., 56:560 (1991); oligodeoxynucleotides as Antisense Inhibitors of Gene Expression, CRC Press, Boca Raton, Fla. (1988)).

Thus advantageously the expression of each of the relevant proteins may be inhibited using antisense technology which may be used to selectively confirm the action of candidate compounds which may be identified as potential treatments for Alzheimers or other neurodegenerative diseases mediated by Tau protein using the transgenic non-human animal described herein, which expresses said human Tau and/or said protein capable of modulating human Tau protein.

Recently it has become possible to manipulate the expression of genes in animals by engineering genetic switches in the genome of the animal which can be designed to target expression or ablation of any gene to any tissue at any defined time. (Inducible gene targeting in mice using the Cre/lox system, a companion to methods in enzymology 14, 381-392 (1998). Using this technology expression of any of the proteins according to the invention can be manipulated, for example, such that expression only occurs when the transgenic line has been established. Accordingly, the vectors of the invention may include a stop signal or sequence between the sequence capable of directing expression of said human Tau or the protein capable of modulating human Tau protein, which stop signal is flanked by two loxP sites. When the vector is used to establish the transgenic line as described above and in the examples below, expression of the relevant protein will not occur unless the Cre recombinase protein is present. The Cre protein catalyses reciprocal conservative DNA recombination between the pairs of loxP sites with the resulting excision of the stop sequence located between the loxp sites. The Cre protein may itself be expressed in another transgenic animal which is mated with the first, to remove the stop sequence following the reciprocal combination event between the two loxP sites to switch on expression of the appropriate sequence in the transgenic animal. This technique also permits the DNA sequence encoding the proteins according to the invention to be excised by the Cre protein by including in the appropriate nucleic acid vector loxP sites flanking the sequences encoding human Tau and/or the protein capable of modulating human Tau protein. Such vectors can be used to investigate the role of null mutations or knock-outs of the sequences encoding the proteins in the transgenic animal according to the invention.

In accordance with the present invention, a defined nucleic acid includes not only the identical nucleic acid but also any minor base variations including in particular, substitutions in cases which result in such as for example a synonymous codon (a different codon specifying the same amino acid residue) due to the degenerate code in conservative amino acid substitutions. The term “nucleic acid sequence” also includes the complementary sequence to any single stranded sequence given regarding base variations.

A further aspect of the invention comprises a method of making a transgenic non-human animal which expresses a human Tau protein comprising the steps of: (a) introducing into an embryo cell of said animal a nucleic acid vector according to the invention; (b) introducing the embryo from step (a) into a female animal; (c) sustaining the female in step (b) until such time as the embryo has sufficiently developed and is borne from the female; and (d) sustaining the transgenic animal.

A further method of generating a transgenic non-human animal which expresses a human Tau protein comprises the steps of (a) introducing sequentially or simultaneously into an embryo cell of said animal a nucleic acid vector comprising a transgene encoding said human Tau protein; and a nucleic acid vector comprising a sequence of nucleotides which upon integration into the genome of said animal are capable of preventing expression of endogenous Tau protein from said animal; (b) introducing the embryo from step (a) into a female animal; (c) sustaining the female in step (b) until such time as the embryo has sufficiently developed and is borne from the female; and (d) sustaining the transgenic animal.

Another method of generating a transgenic non-human animal which is a model for diseases such as Alzheimers disease, comprises crossing a first transgenic non-human animal expressing human Tau protein from a vector according to the invention with a second transgenic non-human animal expressing a protein capable of modulating human Tau protein according to the invention, selecting among the progeny those that carry both expression of said human Tau protein and said protein capable of modulating human Tau protein.

As described above, the Cre/lox technology can be used to manipulate expression of the proteins in each of the transgenic non-human animals described herein by incorporation of loxP sites flanking an appropriate DNA sequence. The sequence may be one or both of those encoding either human Tau or the protein capable of modulating human Tau protein themselves or alternatively a stop sequence or codon which prevents expression of the above proteins unless a recombination event occurs in the presence of Cre recombinase to remove the stop sequence. The vectors used according to this aspect of the invention, to generate the transgenic non-human animals, are incapable of replication in yeast.

A further aspect of the invention comprises a transgenic non-human animal that is a model for Alzheimers disease or for another neurodegenerative disease, which animal comprises an introduced DNA sequence encoding and capable of expressing the protein Tau in the nervous system of said animal and also comprises a DNA sequence encoding and capable of expressing a protein capable directly or indirectly of modulating the human Tau protein. In this aspect of the invention the human Tau and the protein capable of modulating human Tau are preferably those encoded by the sequences on the vectors according to the invention as described above.

A further aspect of the invention comprises a method of generating a transgenic non-human animal which is a model for Alzheimers disease or related neurodegenerative disorders, comprising the steps of crossing a first transgenic non-human animal comprising a vector having, i) a nucleic acid sequence encoding a human Tau protein, ii) a sequence capable of directing expression of said human Tau protein in the nervous system of said animal and iii) a targeting sequence which facilitates integration of said vector into the genome of said animal, with a second transgenic non-human animal comprising a vector capable of expressing a protein capable of modulating human Tau protein according to the invention, selecting among the progeny those that express both human Tau protein and said protein capable of modulating human Tau protein.

Another transgenic non-human animal according to the invention is also provided by crossing a first transgenic non-human animal expressing human Tau protein with another non-human animal transgenic for the protein which modulates human Tau protein. Therefore, according to this aspect of the invention there is provided a method of generating a transgenic non-human animal which is a model for Alzheimers disease or related neurodegenerative disorders, comprising the steps of crossing a first transgenic non-human animal comprising a vector having, i) a nucleic acid sequence encoding a human Tau protein, ii) a sequence capable of directing expression of said human Tau protein in the nervous system of said animal and iii) a targeting sequence which facilitates integration of said vector into the genome of said animal, with a second transgenic non-human animal comprising a vector according to the invention, selecting among the progeny those that express both human Tau protein and said protein capable of modulating Tau protein.

The term “progeny” or “offspring” is intended to include the resulting product of a mating between the transgenic animals described provided it carries a vector according to the invention. Also included are germ cells from said transgenic animals which may themselves be used to produce further offspring comprising a vector according to the invention stably integrated into its genome.

Preferably, the non-human animal used in accordance with the methods of the invention is a mammal and even more preferably a mouse.

The nucleic acid vectors described can be introduced into the embryonic stem cells, by for example electroporation. Microinjection of the cells is performed on the embryo when it is at the one cell stage, thus ensuring that the nucleic acid vector will be incorporated into the germ line of the animal and thus be expressed in all cells of the animals for subsequent transmission to progeny. A further aspect of the invention comprises progeny of the transgenic animal according to the invention, which progeny carries any of the nucleic acid vectors according to the invention stably integrated into their genome.

The transgenic animal may advantageously exhibit the symptoms of Alzheimer's or other related neurodegenerative disorders mediated by human Tau protein phosphorylation making it a suitable model for the disease in humans. Compounds which modulate and interfere with (either by enhancing or inhibiting) the hyperphosphorylation of human Tau protein may be identified by administering the compounds to the animal. Compounds identified as enhancers may advantageously be applied to the animal to enhance development of the disease. Inhibitors of the disease may be identified by monitoring the effects or the phosphorylation profile of Tau protein in the animal following application or administration of the compound to the animal. The compounds may be administered by any suitable route, such as orally or intravenously.

Furthermore, the present invention provides a method of producing a compound which modulates the human kinase mediated hyperphosphorylation of human Tau protein comprising the steps of any one of the above described screening methods; and additionally:
(i) synthesising the compound obtained or identified in said method or a physiologically acceptable analogue or derivative thereof in an amount sufficient to provide said modulator in a therapeutically effective amount to a patient; and/or
(ii) combining the compound obtained or identified in
(iii) said method or an analogue or derivative thereof with a pharmaceutically acceptable carrier.

The compounds isolated by the above methods also serve as lead compounds for the development of analog compounds. The analogs should have a stabilized electronic configuration and molecular conformation that allows key functional groups to be presented to the Tau protein or the kinase in substantially the same way as the lead compound. In particular, the analog compounds have spatial electronic properties which are comparable to the binding region, but can be smaller molecules than the lead compound, frequently having a molecular weight below about 2 kD and preferably below about 1 kD. Identification of analog compounds can be performed through use of techniques such as self-consistent field (SCF) analysis, configuration interaction (CI) analysis, and normal mode dynamics analysis. Computer programs for implementing these techniques are available; e.g., Rein, Computer-Assisted Modeling of Receptor-Ligand Interactions (Alan Liss, New York, 1989). Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, N.Y., USA. Furthermore, said derivatives and analogues can be tested for their effects according to methods known in the art; see also supra. Furthermore, peptidomimetics and/or computer aided design of appropriate derivatives and analogues can be used.

It will be appreciated that not every vector, which may otherwise be referred to as a transgene, will function optimally in every cell or animal type. Thus, routine experimentation may be required to identify or establish the best kinase or Tau isoform or promoter sequence for any given cell or animal type.

Antibodies to the protein or polypeptide of the present invention may, advantageously, be prepared by techniques which are known in the art. For example, polyclonal antibodies may be prepared by inoculating a host animal, such as a mouse, with the polypeptide according to the invention or an epitope thereof and recovering immune serum. Monoclonal antibodies may be prepared according to known techniques such as described by Kohler R. and Milstein C., Nature (1975) 256, 495-497. Advantageously, such antibodies may be included in a kit for identifying the human Tau or the kinase in a sample, together with means for contacting the antibody with the sample.

The invention may be more clearly understood from the following exemplary embodiment and by reference to the accompanying Figures.

Transgenic GSK-3β[S9A] Mice

Five independent transgenic founders were generated that contained the human GSK-3β[S9A] mutant kinase under the control of the mouse thy-l gene promoter, in the FVB genetic background. All experiments were comparatively performed with heterozygous mice from lines GS-3β[S9A]-5 and -1 in which GSK-3β expression was highest and which were concordant in all phenotypic aspects.

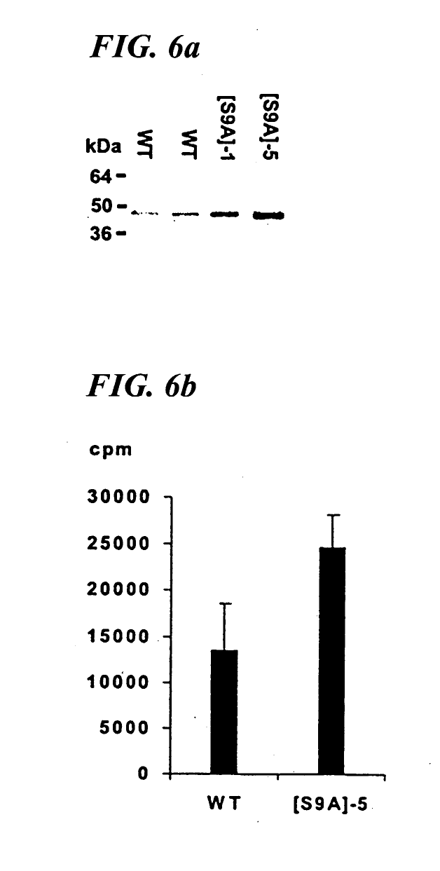

The human GSK-3β protein was revealed by Western Blotting (FIG. 6A) and was enzymatically active towards a GS-1 synthetic peptide. In brain homogenates of transgenic ice, GSK-3β kinase activity was about doubled relative to the activity in wild-type mouse brain (FIG. 6B). Immunohistochemically, the human protein was localized in neuronal cell bodies and in processes in the cortex and hippocampus conform to and expected from the known expression pattern of the adapted mouse thyl gene construct used (Moechars et al., 1996 and references therein).

Transgenic Human Tau40 Mice

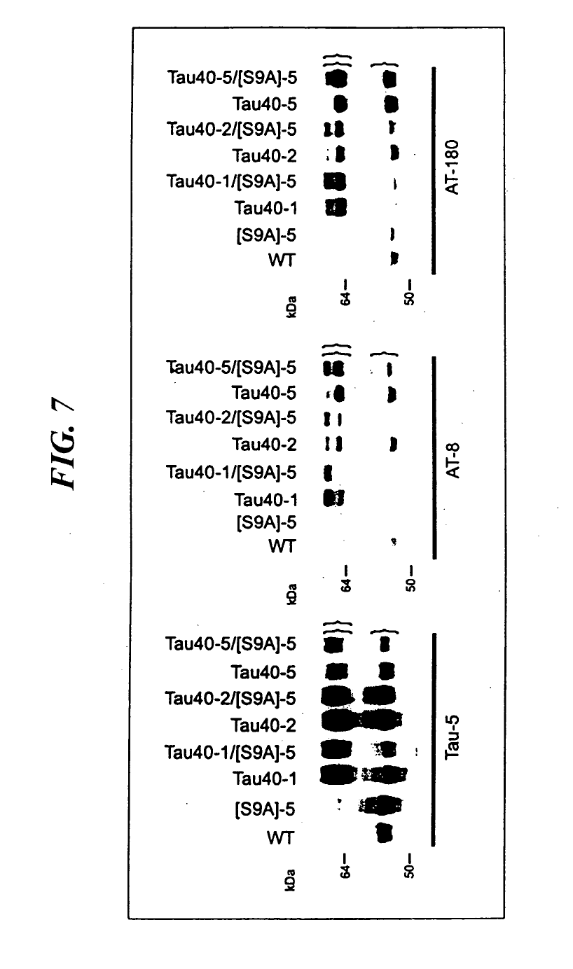

Five independent transgenic founders were generated that contained the human tau40 cDNA, embedded in the adapted mouse thy-1 gene promoter; similar to the construct used above. Three founder lines, i.e. htau40-1, -2 and -5 transmitted the transgene in a mendelian pattern and were analyzed. Western Blotting with the human specific phosphorylation-independent monoclonal antibody HT-7 demonstrated highest expression of human protein tau in lines htau40-1 and htau40-2 (FIG. 3A). Western blots of total protein tau with the phosphorylation-independent monoclonal antibody Tau-5 were quantified by densitometric scanning to demonstrate that the ratio of transgenic human to endogenous mouse protein tau was about 1.5, 1.6 and 0.5 respectively in the three transgenic lines (FIG. 3B). In the brain of human protein tau40 transgenic mice of 4 to 8 weeks old, the antibody HT-7 stained the pyramidal nerve cell bodies and their processes in the hippocampus and the cortex (FIG. 7C), while strong labelling was also evident in cortical layer V.

Transgenic GSK-3β[S9A] and Double [GSK-3β×tau-4R] Mice

The transgenic mice expressing the longest human tau-4R isoform have been described and characterized (Spittaels et al, 1999).

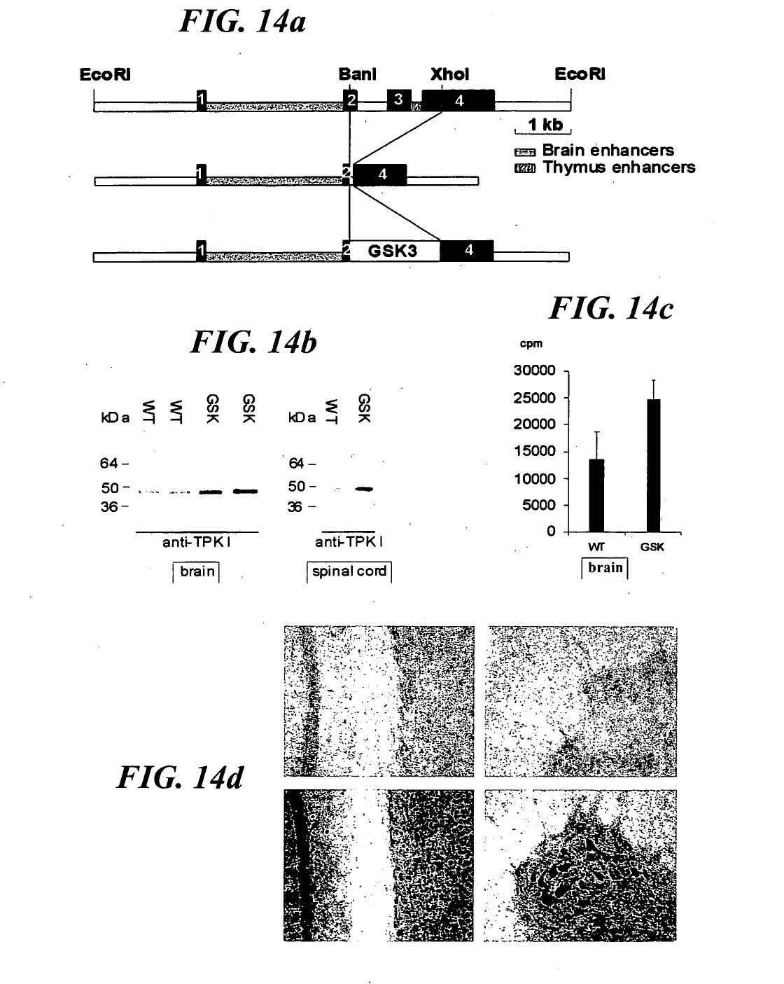

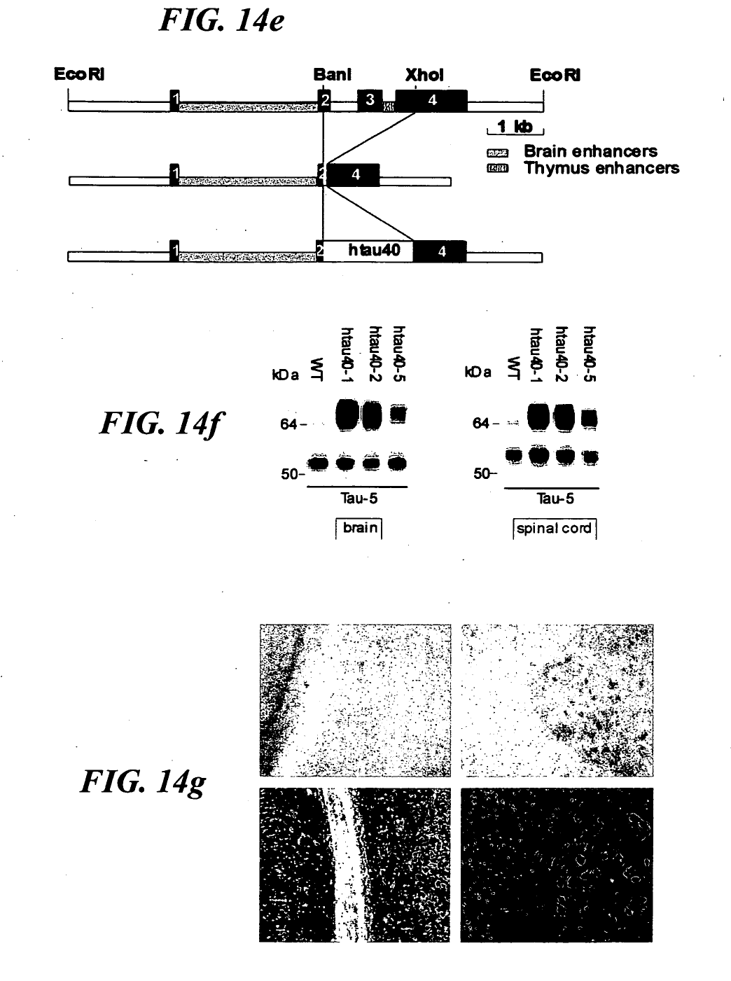

The present inventors have generated transgenic mice that express a mutant form of human GSK3β, denoted GSK-β[S9A] since the cDNA contained an alanine residue in position 9, instead of the wild-type serine to prevent inactivation by phosphorylation (Woodgett, 1990). The cDNA was incorporated in a recombinant DNA construct based on the mouse thy-1 gene promoter (FIG. 14a) and transgenic mice were generated by micro-injection, in the FVB mouse strain (Moechars et al, 1996, 1999; Spittaels et al, 1999).

The human GSK-3β protein was demonstrated by Western blotting in brain and spinal cord (FIG. 14b). The transgene was enzymatically active on a synthetic peptide substrate, resulting.in a doubling of the total GSK-3β kinase activity in GSK-3β mouse brain homogenates, relative to wild-type mice (FIG. 14c). Immuno-histochemically, the human transgenic proteins were localized in neuronal cell bodies and in processes in the cortex and hippocampus (FIG. 14d), as expected for the adapted mouse thyl gene construct (Moechars et al., 1996, 1999; Spittaels et al, 1999). In addition, motor neurons in the ventral horn of the spinal cord also expressed the human GSK-3β [S9A] mutant and the human tau transgene as well (FIG. 14d, g) (Spittaels et al, 1999). Both transgenes were thus demonstrated to be expressed in the same neurons, in the same regions of brain and spinal cord.

Double transgenic mice were obtained by cross-breeding the single transgenic strains.

Analysis of Phosphorylation of Protein Tau

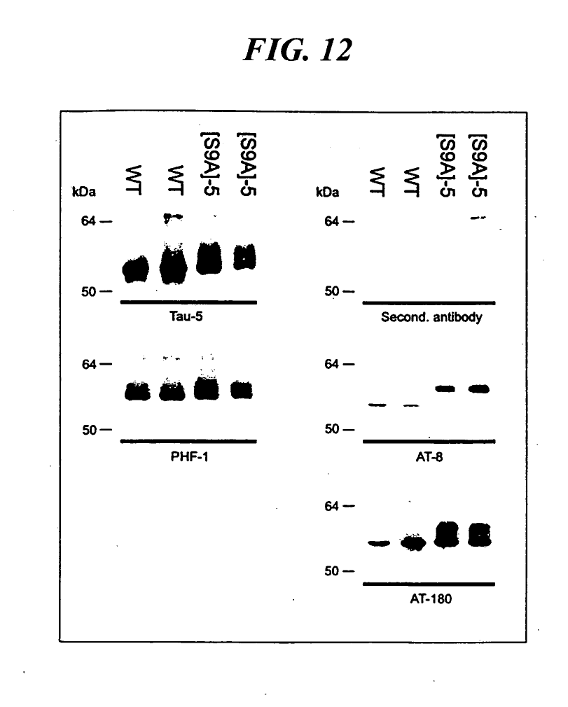

Extensive analysis was performed by Western Blotting of mouse brain extracts with a battery of well-characterised antibodies including antibodies specific for different epitopes of protein tau known to be phosphorylated by GSK-3β (Sperber et al., 1995). In GSK-3β[S9A] transgenic mice, 4-8 weeks old, only minor differences in electrophoretic migration was observed relative to age-matched wild-type mice and analyzed by Western Blotting with the PHF-1 antibody. The hyperphosphorylation of murine protein tau in such transgenic animals was evidenced by AT-180 immunoreactivity, but only by longer exposure of the western blots. The additional, and wanted reaction with antibody AT-8 remained, however, absent in the single transgenic mice of less than 2 months old. In the brain of older GSK-3β transgenic mice, i.e. aged up to 16 months (FIG. 12), the immunoreactivity of both phosphorylation-dependent antibodies AT-8 and AT-180 were observed clearly, concomitant with a slower electrophoretical mobility of the immuno-reactive isoforms of protein tau detected also with monoclonal antibodies Tau-5 and PHF1 (FIG. 12).

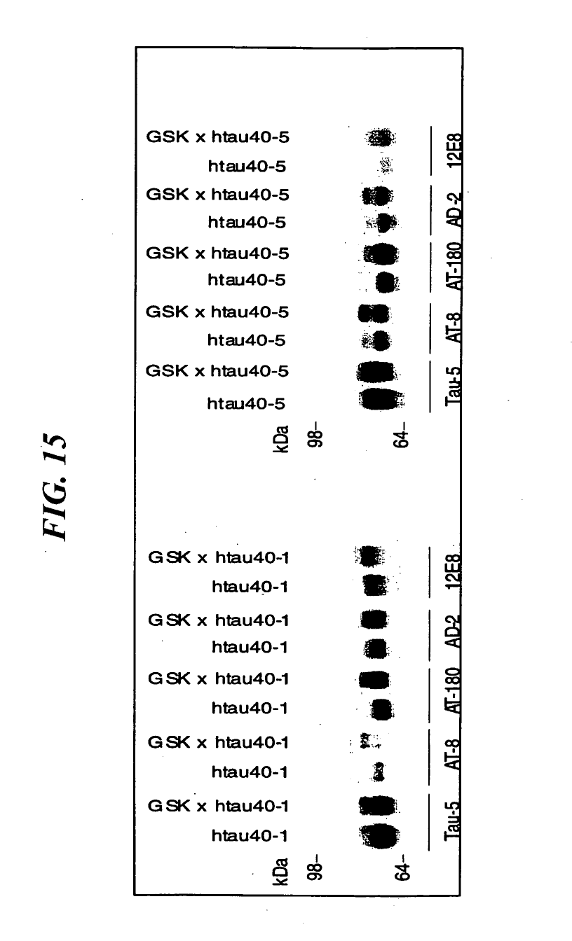

Extensive immuno-histochemical analysis was performed on both paraffin and on cyrostat sections cut from brain of mice sacrificed at different ages and processed and fixed following several different procedures. Staining with the antibodies used in Western Blotting and with many additional antibodies failed to reveal appreciable and reproducible phosphorylation of endogenous mouse protein tau in the GSK-3β transgenic mice. It is obvious that human protein tau is far better characterized than mouse protein tau, and that the antibodies used are primarily directed to human protein tau. Since, moreover, evidence for any tau-pathy is lacking in mice, the present inventors decided to investigate the role of GSK-3β in mediating phosphorylation of human protein tau in vivo, by generating double transgenic mice coexpressing human protein tau next to GSK-3β[S9A] in the same neurons. Definite hyper-phosphorylation of human protein tau was demonstrated in brain extracts of these double transgenic mice, even at the early age of 5 weeks, by Western Blotting with antibodies AT-8, AT-180 and Tau-5 (FIG. 7). Brain of single transgenic littermates expressing GSK-3β or human tau40 only, showed no or much weaker AT-8 and AT-180 immunoreactivity and contained no or much less of the slower migrating isoforms of protein tau. By the same western blot-methods, hyper-phosphorylation of human protein tau was demonstrated in brain extracts of double tau4R×GSK-3β[S9A] transgenic mice, even as young as 5 weeks. Western blotting with antibodies AT-8, AT-180, AD-2 and 12E8 reacted with slow migrating human protein tau isoforms in brain homogenates of double transgenic mice, and these were virtually absent in their single transgenic littermates (FIG. 15).

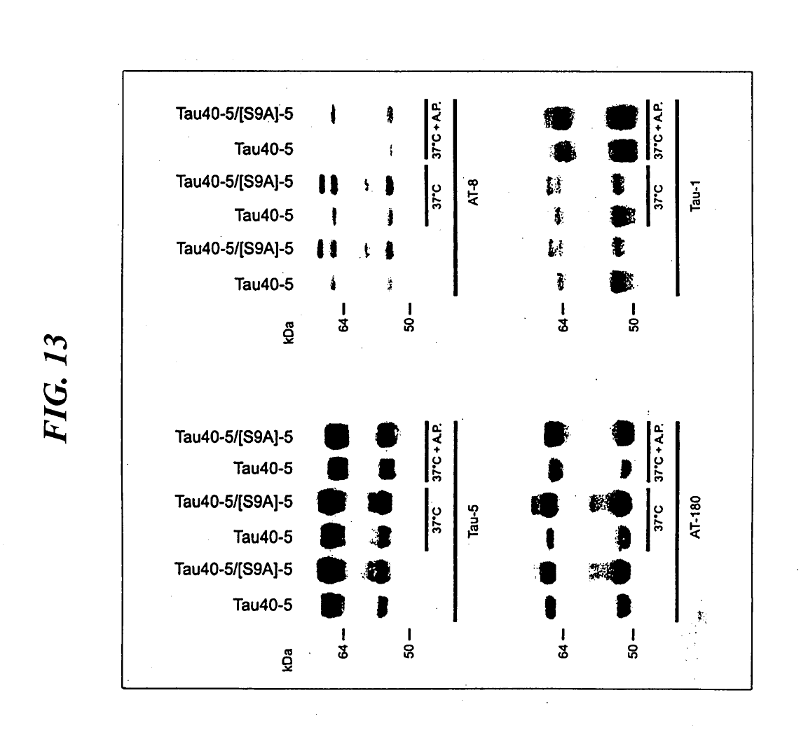

Pre-treatment of brain extracts with alkaline phosphatase prior to electrophoresis, yielded identical protein tau patterns of all mice on the Western Blots and abolished AT-8 and AT-180 immunoreactivity of both murine and human protein tau (FIG. 13). In addition, prior de-phosphorylation increased immunoreactivity with antibody Tau-1 (FIG. 13).

GSK-3β Reduced the Amount of Protein Tau Bound to Microtubules

We examined whether GSK-3β activity affects protein tau binding to microtubules in brain and spinal cord extracts. Cytoplasmic extracts, isolated in the presence of taxol still can perform and assemble into microtubular structures, despite the unfavourable conditions of low tubulin concentrations, the presence of proteases and other harsh conditions. Such re-assembled preparations still allow association of MAP with microtubules (Vallee 1982).

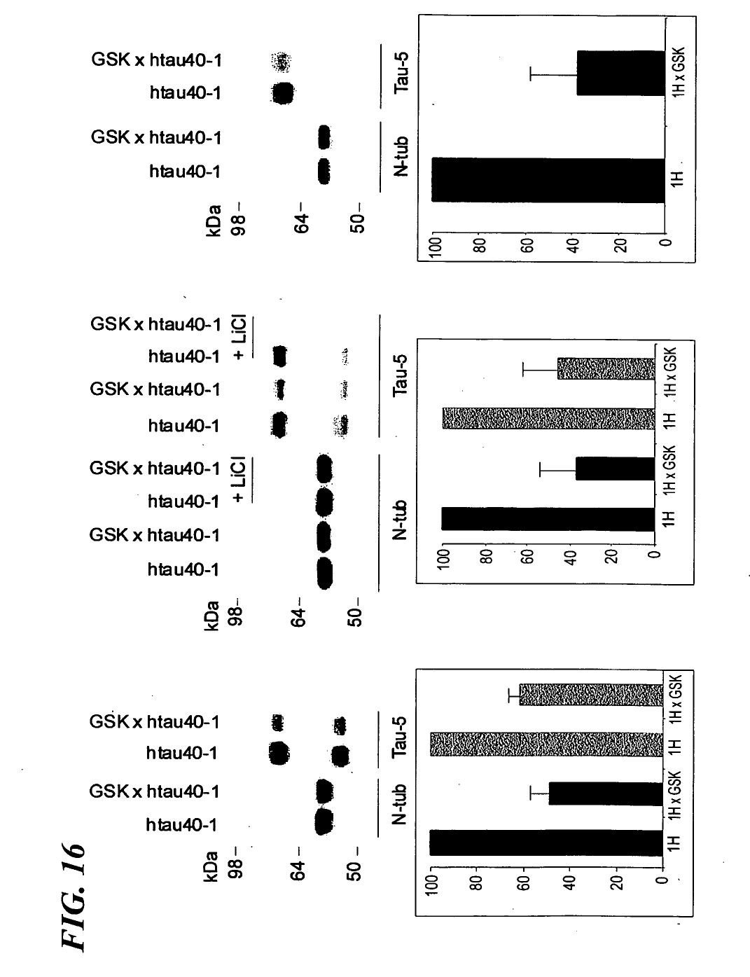

The binding of protein tau to re-assembled microtubules extracted from mouse brain and spinal cord, was significantly reduced in homogenates derived from htau4R×GSK-3β double transgenic mice, compared to htau4R littermates (FIG. 16). The presence of phosphatase inhibitors was essential while addition of LiCl during the isolation procedure did not affect this result, indicating that the tau phosphorylation had occurred in vivo and reflected the condition as was in the brain of the htau40-1×GSK-3β transgenic mice (FIG. 16).

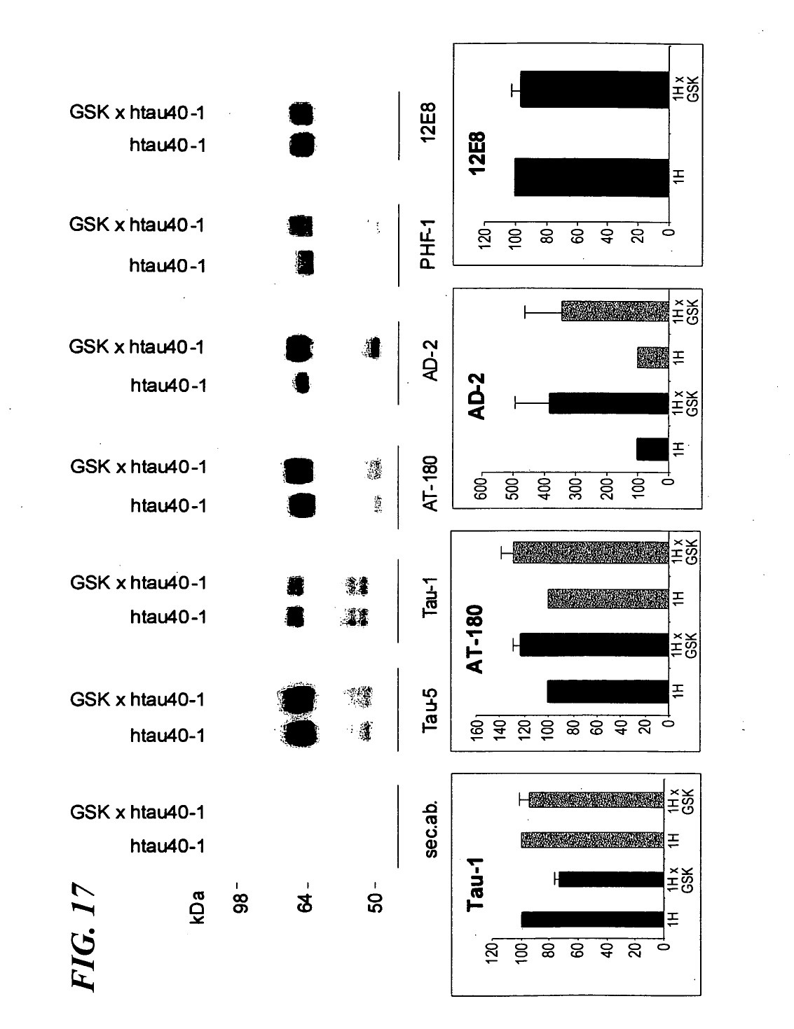

Reduced binding to microtubules was evidently related to hyperphosphorylation as demonstrated by analysis of unbound protein tau, human and murine, that remained in the supernatant of these extracts. This soluble protein tau was hyper-phosphorylated as evidenced by reaction with antibodies Tau-1, AT-180 and AD-2 (FIG. 17). Quantitatively most reactive was the epitope recognized by AD-2, corroborated by reaction with PHF-1 antibody that recognizes the same phosphorylated epitope on protein tau.

Quantitative analysis by densitometric scanning and normalization to the reaction with antibody Tau-5, demonstrated an almost 4-fold increase of phosphorylation at the epitope defined by these monoclonal antibodies (FIG. 17). Antibodies AT-180 and Tau-1 revealed only a moderate increase in the phosphorylation of their respective epitopes in protein tau in the double transgenic mice, relative to the single transgenic tau4R mice.

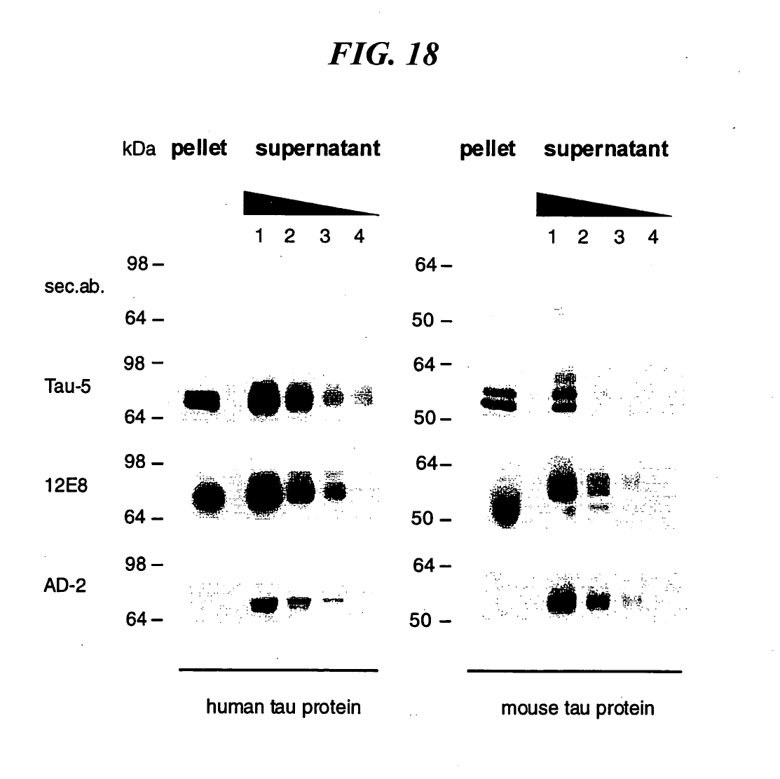

Next, we demonstrated that the AD-2 and 12E8 epitopes are differentially present on the bound and free protein tau in the microtubule extracts. Western blotting revealed that the 12E8 epitope was detectable on bound and free protein tau, as opposed to the AD-2 phospho-epitope which was not detectable in the microtubule-associated protein tau (FIG. 18).

The epitopes of AD-2 and 12E8 encompass Ser³⁹⁶/Ser⁴⁰⁴ (Buee-Scherrer et al., 1996) and Ser²⁶²/Ser³⁵⁶ (Seubert et al., 1995), respectively, and have been discussed as pivotal in the tau-microtubule interaction, subject to regulation by phosphorylation (Bramblett et al., 1993; Sengupta et al., 1998). Our results suggest that phosphorylation of the AD-2 epitope, in our conditions by GSK-3β, could indeed be essential for this interaction.

Co-Expression of GSK-3β Reduced the Axonopaty in CNS of htau40 Transgenic Mice

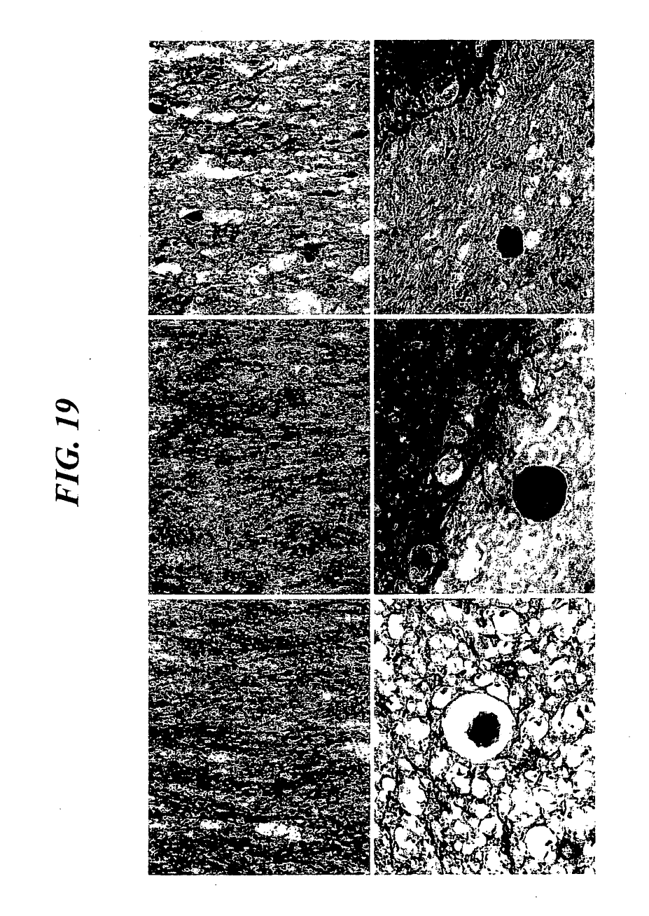

A pathological hallmark of the tau4R transgenic mice, i.e. the presence of dilated axons (Spittaels et al., 1999) is now demonstrated to contain synapthophysin-bearing vesicles, normally rapidly transported to the synapse by the motor protein kinesin, which also accumulated in the diseased axons (FIG. 19). Consequently, the excess protein tau appeared to inhibit axonal transport by binding to the microtubules in the tau4R transgenic mice, and this then caused the axonal dilatations and the axonopathy (Spittaels et al, 1999).

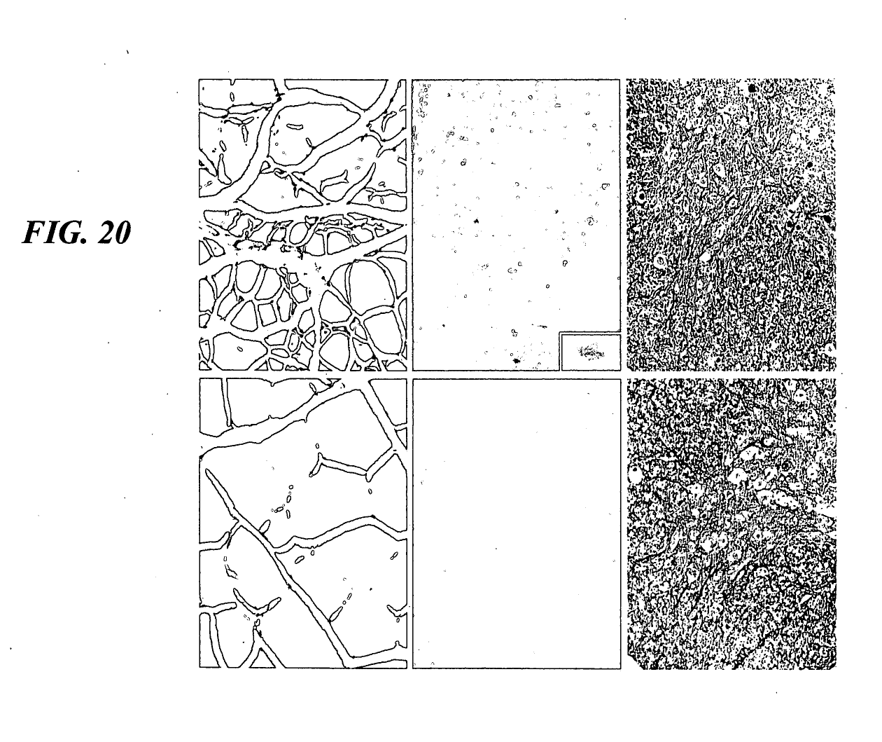

In the brain and spinal cord of double tau4R×GSK3β transgenic mice, the number of dilated axons was dramatically reduced (FIG. 20, Table 1). The same result was. observed in double transgenic mice obtained with a different parental trau transgenic strain (Spittaels. et al, 1999) and denoted as htau40-2×GSK-3β (Table 1). Concomitantly, the grouping of atrophic fibers and the fascicular atrophy in htau40 transgenic mice, diagnostic for their neurogenic atrophy, was dramatically reduced in the double transgenic mice. The quadriceps of htau40-1×GSK-3β mice was devoid of any muscle wasting that is a pathological hallmark in the htau40 animals (FIG. 20) (Spittaels et al,1999).

Co-Expression of GSK-3β Rescued the Motorical Impairment of htau40 Transgenic Mice

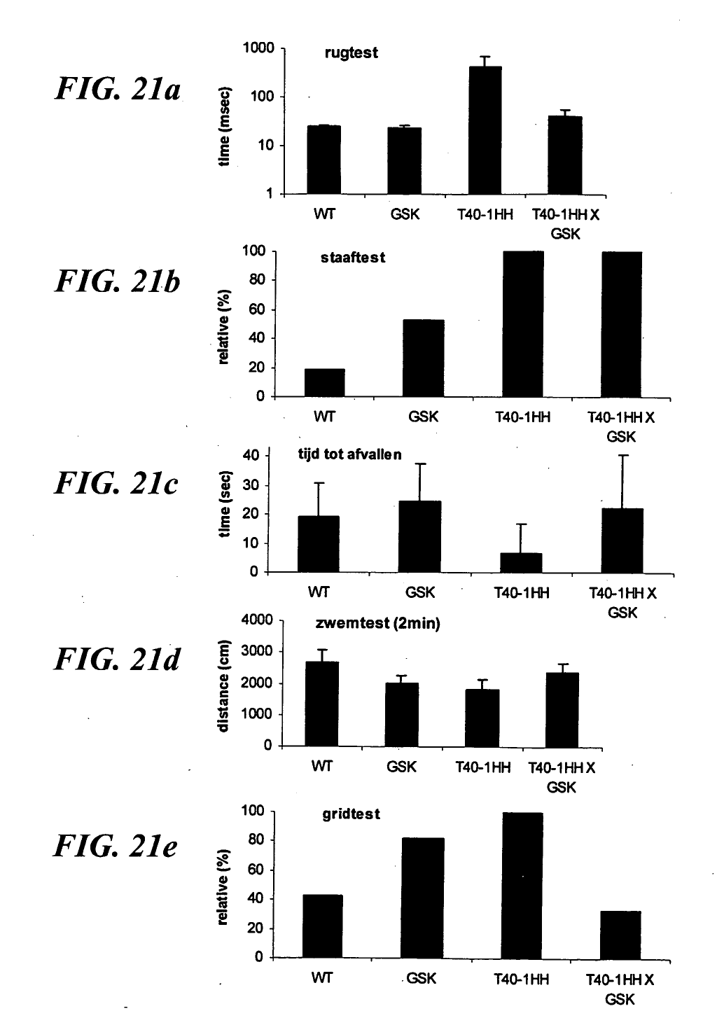

The axonopathy and the severe motor problems were both directly correlated to the level of expression of the human tau4R transgene protein, in 3 different htau4R transgenic founder strains (Spittaels et al., 1999). The effect of co-expression of GSK-3β on the motoric aspect of the phenotype, was evaluated by five different tests, in double htau40×GSK-3β transgenic mice, relative to htau40-1, GSK-3β and wild-type mice (FIG. 21).

Overall, the double tau4R×GSK-3β transgenic mice behaved in all tests significantly better than their single parental strains, with the exception of one parameter in the rod-walking test (FIG. 21). Interestingly, the tests revealed important characteristics of the single transgenic mice as well.

In the “uprighting reflex”, the time needed to return when forced to lay on their back (FIG. 21a), the evident impairment of the single tau-4R transgenic mice was nearly completely corrected in the double htau40×GSK-3β transgenic mice (FIG. 21a).

In the forced swimtest (FIG. 21d) and in the grid-hang test (FIG. 21e) the double transgenic mice performed as good as wild-type mice and significantly better than single htau40 and single GSK-3β mice (FIGS. 21b, e).

Most difficult to interpret was the rod-walking test in which two independent parameters were measured: the number of mice that dropped of the rod (FIG. 21b) and the time they remained on the rod (FIG. 21c). The first parameter clearly did not differentiate between the single tau4R and double transgenic mice, demonstrating that all the tau-4R transgenic mice were unable to remaining on the rod, a characteristic not affected by GSK-3β co-expression. On the other hand, the time that the mice remained on the rod was restored to that of the wild-type mice in the double transgenic mice, as opposed to the markedly reduced time in the single tau4R transgenic mice (FIG. 21c). Since both parameters reflect different aspects of behaviour, motoric capacity and ability, it will be interesting to analyze this further by other means in depth and with the additional effect of ageing.

Interestingly, although the GSK-3β transgenic mice as such, displayed reduced motor ability in the three psycho-motoric tasks relative to wild-type mice, the double transgenic mice were always more successful than the single htau40 mice. These observations demonstrate that expression of GSK-3β already affected the motoricity of the transgenic mice, but that its co-expression with human protein tau rescued to a large extend or even completely, the phenotype of the tau-4R transgenic mice, as measured by endurance, postural stability, motor coordination, equilibrium maintenance and muscle strength.

TABLE 1
Quantification of dilated axons in brain and
spinal cord of transgenic mice

| | |
|---|---|
| Spinal cord | Cerebral cortex |

| Mouse type | n | Mean no.* | n | Mean no.** |
|---|---|---|---|---|
| WT | 4 | 0 | 4 | 0 |
| GSK | 4 | 0 | 4 | 0 |
| htau40-1 H | 8 | 9.9 | 8 | 10.7 |
| htau40-1 H × GSK | 3 | 2.2 | 4 | 0.5 |
| htau40-2 H | 8 | 3.6 | 4 | 2.6 |
| Htau40-2 H × GSK | 4 | 0.3 | 4 | 0.3 |

Numbers of dilated axons in entire transversal sections of the spinal cord (6 μm thick) and in coronal sections of the entire right hemisperic cortex (40 μm thick) are presented. Numbers of dilated axons in the cerebral neocortex from sections through the hippocampus were counted. Silver impregnation (9 sections per mouse type counted) and SMI-32 immunostaining (8 sections per mouse type counted) yielded similar results. Mice used were three months of age.

The Kruskal-Wallis analysis revealed a significant decrease of the number of axonal dilations in both the spinal cord and hemispheric cortex of htau40×GSK double transgenic mice compared to htau40 single transgenic littermates. (*) htau40-1H-htau40-1 H×GSK: p=0.0181; htau40-2 H-htau40-2 H×GSK: p=0.0073. (**) htau40-1H-htau40-1H×GSK: p=0.0063; htau40-2 H-htau40-2 H×GSK: p=0.017.

WT denotes wild-type mice, n: number of mice analyzed.

The hypothesis that GSK-3β is a major kinase capable of hyperphosphorylation of protein tau in brain was first approached and tested in vivo, by overexpression of a constitutively active human kinase, i.e. GSK-3β[S9A] in the brain of transgenic mice using the mouse thyl gene promoter. The transgene was enzymatically active in brain and expressed mainly in hippocampal and cortical neurons, thereby about doubling the overall GSK-3β kihase activity. Murine protein tau extracted from the brain of young GSK-3β[S9A] transgenic mice was somewhat hyperphosphorylated, as manifested by the presence of isoforms with slower electrophoretic migration, with some AT-180 immunoreactivity but weak or absent AT-8 reaction on western blots. In older mice tested at 7 and 16 months of age, endogenous protein tau isoforms with clearly retarded electrophoretic mobility and with strong AT-8 immuno-reactivity were evident in the brain. Isoforms of murine protein tau that migrated on 8% polyacrylamide gels as a broadened band, reacted with antibodies PHF-1 and Tau-5. The increased PHF-1 immunoreactivity caused by GSK-3β-mediated hyperphosphorylation can be attributed to phosphorylation of serine residues at positions 396 and/or 404 that participate in this epitope (Otvos et al. 1994). In vitro studies showed that hyperphosphorylation of these and other epitopes rendered the slower migrating tau isoforms.

The reasons to investigate not only endogenous mouse protein tau but also human protein tau as substrate for GSK-3β[S9A] in these transgenic mice, are many and not only practical. Evidently, all the typical and specific antibodies used to detect phosphorylated epitopes on protein tau are directed against the human protein. In addition, evidence for any involvement of endogenous murine protein tau in tau-pathy is lacking.

Therefore, the ability of GSK-β3 to mediate phosphorylation of human protein tau was investigated by generating double transgenic mice, i.e. mice that coexpress the human tau40 protein isoform and the human GSK-3β[S9A] mutant kinase. To this end, transgenic mice were generated that overexpressed the longest human protein tau isoform, i.e. human protein tau40 containing 2 N-terminal inserts and 4 microtubule binding repeats. Using the same type of gene promoter construct assured the expression of both transgenes to coincide inside the same neurons in brain. In the single and double transgenic mice, human tau protein accounted for up to 60% of total protein tau in the brain of the highest expressing transgenic mouse line.

Immunodetection with HT-7 revealed a somatodendritic localisation in addition to axonal staining, similar to a previous report on human tau transgenic mice (Götz et al. 1995), and resembling the localisation of endogenous protein tau in central neurons (Tashiro et al. 1997).

The cross-breeding yielded the expected numbers of double transgenic mice offspring, which were identified by genotyping and demonstrated by Western Blotting to co-express human protein tau40 and human GSK-3β[S9A]. In the brain of the double transgenic mice, unambiguous and robust hyper-phosphorylation of protein tau was evident as early as 5 week-old of age, by the presence of slower migrating isoforms reacting strongly with both antibodies AT-8 and AT-180. This proved that the epitopes of antibody AT-8, involving serine 199 and/or 202 residues and the epitope of antibody AT-180, involving threonine 231, were abundantly phosphorylated, not excluding additional phosphorylation at other residues to induce the slower migrating tau proteins. Tau=s binding to microtubules was eliminated by the phosphorylation of several sites (Mandelkow et al. 1995, Trinczek et al. 1995, Preuss et al. 1997), among which residue Thr231 was of major importance (Sengupta et al. 1998). De-phosphorylation prior to electrophoresis destroyed both the AT-8 and AT-180 immunoreactivity, increased the reaction with antibody Tau-1 and increased the electrophoretic mobility of protein tau.

The cDNA coding for human GSK-3β[S9A] (Sutherland et al. 1993; Stambolic and Woodgett, 1994) was ligated in the mouse thyl gene (Moechars et al., 1996). A PvuI-NotI restriction fragment was micro-injected into 0.5 day old FVB/N prenuclear mouse embryos. Transgenic founders were identified by southern blotting of Stul-restricted mouse tail-biopt DNA, hybridized with a probe of 701 bp obtained by PCR with forward primer 5′CAAGGTCCCCGTTTCTCC3′ (SEQ ID NO :1) and reverse primer 5′CAGGGGATAGTGGTGTGG3′ (SEQ ID NO:2). Routine genotyping of transgenic offspring, bred into the FVB/N genetic background, was performed on tail-biopt DNA with forward primer 5′CCCCACCACAGAATCCA3′ (SEQ ID NO:3) located in the mouse thyl gene and with reverse primer 5′GCTGCCGTCCTTGTCTCT3′ (SEQ ID NO:4) located in the human GSK-3β cDNA. Human Tau40 was ligated in the mouse thyl gene. A Pvul-Notl restriction fragment was micro-injected and transgenic founders identified by southern blotting of Stul-restricted mouse tail-biopt DNA. The probe of 135 bp was obtained by PCR with forward primer 5′CCCCACCACAGAATCCA3′ (SEQ ID NO:10) located in the mouse thyl gene and reverse primer 5′GCCCCCCTGATCTTTCC3′ (SEQ ID NO:5) located in the human tau40 cDNA. Routine genotyping of transgenic offspring, bred into the FVB/N genetic background, was performed on tail-biopt DNA by PCR with a forward primer 5′CTGGGGCGGTCAATAAT3′ (SEQ ID NO:6) located in the human tau40 gene and a reverse primer 5′CAAGGTCCCCGTTTCTCC3′ (SEQ ID NO:11) located in the mouse thyl gene, yielding a 213 bp amplicon. GSK-3β[S9A] protein levels in brain extracts were estimated by Western Blotting with monoclonal antibodies TPK I/GSK-3β (0.1 Fg/ml) and htau40-protein levels with monoclonal antibodies HT-7 (0.5 Fg/ml) and Tau-5 (0.5 Fg/ml). Kinase enzymatic activity was measured on brain homogenates after immunoprecipitation and fractionation by ion-exchange FPLC (Mono S) (Pharmacia, Uppsala, Sweden) (Van Lint et al. 1993).

For immunohistochemistry of brain, mice were anesthetized with nembutal and intracardially perfused with either paraformaldehyde (4% v/v) or methacarn (MC) (50% methanol, 30% chloroform, 10% acetic acid). Brains were immersion-fixed overnight, dehydrated and embedded in paraffin (unless stated otherwise). Microtome sections (6 Fm) were dewaxed, hydrated and incubated with blocking solution, i.e. 3% BSA, 10% normal goat serum in Tris Buffered Saline (TBS) (50 mM Tris, pH7.4, 0.15 M NaCl). Incubation was for 12 hours with primary antibodies and for 1 hr with biotin conjugated secondary antiserum in blocking solution, and immunoreactivity was intensified with the Strep-ABComplex/HRP system (Dako A/S, Denmark). A monoclonal antibody to MAP2 (1/400) was used to mark dendrites.

For immunohistochemical detection of human tau in the htau4o transgenic mice, paraformaldehyde (4% in PBS) fixed free-floating vibratome slices (40 Fm) were subsequently incubated with 300 Fl blocking solution (see above) for one hour and overnight with 250-300 Fl primary antibody (HT-7, 2.5 Fg/ml; AT-8, 2.5 Fg/ml; AT-8, 2.5 Fg/ml; PHF-1, 1/50) in blocking solution in a 24 well Costar cell culture plate. Next, brain sections were rinsed with 500 Fl TBS (3×5′), incubated with biotin conjugated secondary antibody (1/1000) for one hour, washed (3×5′) and pretreated with 500 Fl 0.05 M Tris-HCl for 5 minutes. These tissue sections were submerged in 300 Fl Strept-ABComplex/HRP (1 droplet of both solutions per 15 ml 0.05 ml 0.05 M Tris-HCl) for half an hour and successively washed (3×5′), pretreated with 500 Fl 0.05 M Tris-HCl for 5′ and stained with DAB.

For Western Blotting, brain tissue was homogenized in 2 ml of MES buffer with inhibitors, i.e. 0.1 M MES (pH 6.4), 0.5 mM MgCl₂, 1 mM EDTA, 1 mM EGTA, 1 mM DTT, 5 Fg/ml leupeptin, 5 Fg/ml pepstain, 1 FM okadaic acid, 200 FM PMSF, 20 mM NaF, 200 FM sodium orthovanadate, 5 Fg/ml soybean trypsin inhibitor, 1% Triton-X-100, 1% sodium desoxycholate and 0.1% SDS. After centrifugation (100,000 g for 30′ at 4° C.), portions of the supernatant were denatured and reduced prior to separation on Tris-glycine buffered polyacrylamide gels (8% SDS-PAGE) (Novex, San Diego, Calif.) and transferred to nitrocellulose filters. Following antibodies were used: monoclonal AT-8 and AT-180 (1 Fg/ml), PHF1 (1/25) and Tau-5 (1 Fg/ml). Signals were quantified by densitometry and normalized to signals obtained on the same blots with phosphate-independent antibody Tau-5.

Since mouse immunoglobulins interfere with the AT-8 and AT-180 immunoreactivity on Western Blotting (˜50kDa), brain homogenates of GSK-3β transgenic mice were incubated with immobilized protein-G (Pierce, Ill., USA) at 4° C. for 2.5 hours and purified from mouse IgG by centrifugation (8000 rpm, 5′, 4° C.). The supernatant was denatured and reduced prior electrophoretical separation.

To dephosphorylate the tau protein, brain homogenates were diluted in a dephosphorylation buffer (Boehringer Mannheim) containing alkaline phosphatase (Boehringer Mannheim, 0.5 unit/Fl homogenate) and gently stirred at 37° C. for 3 hours. Samples to be loaded on the gel were prepared as mentioned above.

Antibodies HT-7 (directed to human tau), AT-8 (directed to phosphorylated Serl99 and/or Ser202 (Biernat et al. 1992) and AT-180 (directed to phosphorylated Thr231 (Goedert et al. 1994) are purchased from Innogenetics, Gent, Belgium. Anti-TPKI/GSK-3β was bought from Affinity, Nottingham, UK; Tau-5 (recognizing tau, phosphate-independent) from Beckton Dickinson, San Diego, Calif.; Tau-1 (directed to non-phosphorylated Ser199 and Ser202 (Biernat et al. 1992) from Boehringer Mannheim, Germany and biotin conjugated secondary antiserum from Biorad Labs, Calif. PHF1 (directed to phosphorylated Ser396 and Ser404 (Otvos et al. 1994) was a gift of P. Davies.

Synthesis of the Construct to Target the Mouse Tau Locus

A 1.9 kb Not I fragment encoding the 3′ loxP and the Hygromycin B phosphotransferase gene driven by the phosphoglycerate kinase (PGK promoter) was first cloned into the BamHI site of the pBluescript vector. Secondly, a 8 kb Mlu IβAat II fragment containing the human Tau cDNA coding for the longest isoform of human adult Tau driven by the mouse Thy I promoter was cut out together with a 5′ loxP site from the pGEM lox vector and subcloned into the SmaI site of the Bluescript vector (referred to as the Thy-I Tau 40 construct). From this recombinant vector a 10 kb, Sal I β Not I fragment was introduced into the unique Nco I site of the exon I of mouse Tau gene. Prior to electroporation into the ES cells, this targeting vector was linearised with Not I restriction enzyme and gel purified. The yield of the targeting vector was analysed both by gel electrophoresis and optical density using an UV spectrophotometer, wherein the O.D was measured at 260 nm.

ES Cell Culture, Selection and Genotyping

The ES cell line E14 (Hooper et al., 1987) was cultured on mitomycin-treated STO fibroblasts, in Glasgow Me. medium containing non-essential amino acids, 20% (w/v) fetal calf serum, 0.1 mM 2-mercaptoethanol and 1 mM sodium pyruvate. Trypsinized ES cells (1.5-2×10⁷) were resuspended in 500 Fl of culture medium and electroporated with 10 to 15 Fg of the linearised targeting DNA, using an electric pulser (Biorad Labs.) at settings of 200 V and 960 FF in electroporation cuvettes of 0.4 cm electrode distance. The electroporated ES cells were seeded onto mitomycin treated STO fibroblasts in 25 cm² flasks and 40 hours later, the medium was replaced with medium containing 100 Fg/ml of Hygromycin B. Hygromycin resistant colonies were picked up 10 to 14 days later after electroporation and further expanded for genotyping.

DNA was isolated from the selected ES cell lines and 10 Fg was digested with the desired restriction enzyme for 4 to 6 hours. The digests were separated by electrophoresis at 2 V/cm mechanism for 14 hours on 0.7% agarose gels resulting in an overnight run. The following day the gels were stained by Ethidium bromide and photographed, processed for capillary transfer to nylon membranes. After baking and pre-hybridisation, the blot was hybridised with the radiolabelled probes at a concentration of 2-5×10⁶ cpm/ml and kept overnight at 60° C. Hybridisation was carried out in 6×SSC, 5×Denhardt's solution, 1% SDS, 0.1% heparine, 10% Dextran sulphate and 0.1% Salmon Sperm DNA. Membranes were washed at 60° C. for one hour in 0.3×SSC, 0.5% SDS and placed for autoradiographic exposure at 70° C.

Genotyping by Southern Blotting

For Southern blotting 10 Fg of genomic DNA was digested for 5 hrs at 37° C. and separated by electrophoresis in 0.7% agarose gels. DNA was transferred by capillary transfer to a nylon membrane with 10×SSC (1.5M sodium chloride, 150 mM sodium citrate, pH 7.2). The membrane was baked for 2 hrs at 80EC, pre-hybridised for 6 hrs at 60° C. in 6×SSC, 4×Denhardt's solution, 1% SDS, 100 Fg salmon sperm DNA, 10% dextran sulfate and 0.05% heparin. Hybridisation was carried out overnight at 60° C. in the same solution supplemented with 2-5×10⁶cpm/ml of the indicated [³²P]-labelled DNA probe. The membrane was washed in 0.3×SSPE supplemented with 0.5% SDS for 1 hr at 60° C. before autoradiographic exposure with intensifying screens at −70 C. for 1-7 days.

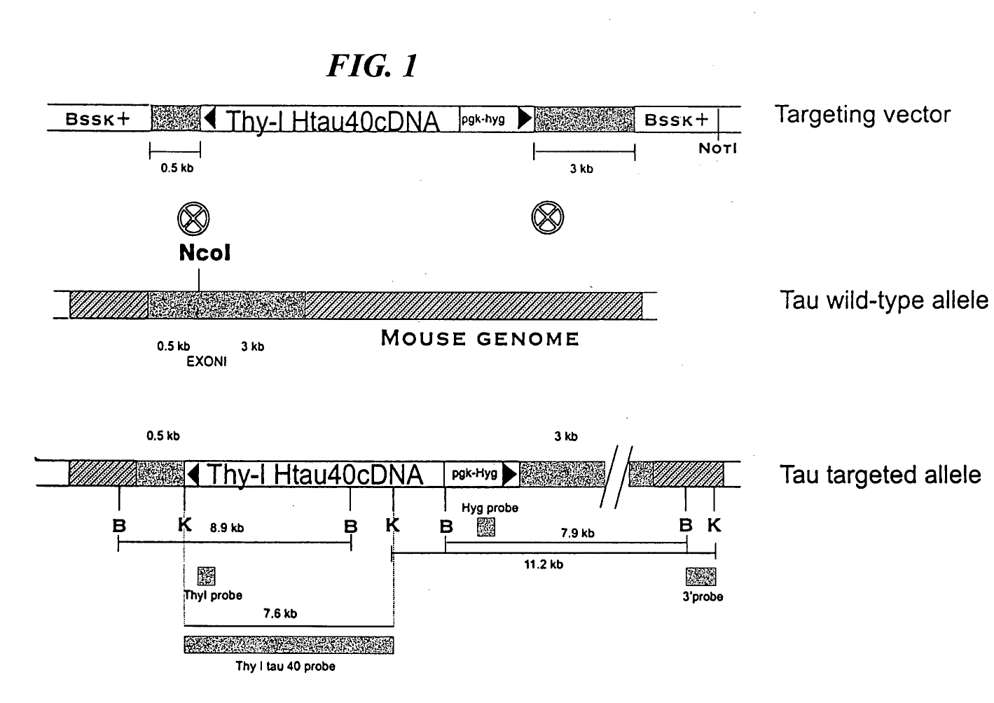

Different probes were designed to genotype the ES cell lines. The ThyI-Tau-40 probe as mentioned above, Hygromycin probe (a gift from Lieve Umans, Lutgarde Serneels and Anton Roebroek) and a 3′ probe. The latter was made by a BamHI-Kpn I restriction of a 13 kb EcorV-Hind III fragment harbouring exon 1 and the intron between the exons 1 and 2 of the mouse Tau gene cloned in the Bluescript vector (gift by Hirokawa,1997) yielding an external probe. The 3′ external probe thus obtained was purified from the gel and used for the first screening of the electroporated ES cells cultured on Hygromycin containing selection medium. Since this probe recognised a region outside the construct, it helped us to figure out whether homologous recombination had occurred or not.(FIG. 1). The ThyI probe used to check the 5′ region of the construct is obtained by ApaI digestion of the Thy I DNA (Prof.Van Der Putten).

Genotyping by PCR

Genotyping for mouse thyI-Tau40 transgenic mice by duplex PCR using the following two sets of primers:
(i) the P16 forward primer in the mouse thyl gene promoter: 5′CCCCACCACAGAATCCA (SEQ ID NO:10) in combination with NE199 reverse primer in the human Tau-40 cDNA, 5′GCCCCCCTGATCTTTCC3′(SEQ ID NO:12), yielding an amplicon of 135 bp;
(ii) the NE200 forward primer in the human Tau-40 cDNA 5′CTGGGGCGGTCAATAAT3′(SEQ ID NO:6) combined with the P62 reverse primer located in the mouse thy-1 gene 5′CAAGGTCCCCGTTTCTCC3′ (SEQ ID NO:l), producing a 213 bp amplicon.

The PCR programme consisted of 30 cycles of denaturation at 95° C. for 1 min, annealing at 60° C. for 1 min and elongation at 72° C. for 15 secs.

Western Blotting

Brain tissues were homogenized in 2 ml of 0.1M MES Buffer pH 6.4, 0.5mM MgCl₂, 1 mm EDTA, 1 mM EGTA, 1 mM DTT, 0.2 mM PMSF, 20 mM NaF, 0.2 mM Na₃VO₄, 1FM okadaic acid, 5 Fg/ml leupeptin, 5 Fg/ml pepstatin, 5 Fg/ml soybean trypsin inhibitor, 1% sodium desoxycholate, 1% Triton-X-100 and 0.1% SDS (Genis et al., 1995, with minor modifications). The brain extract so obtained was denatured at 95° C. for 10 min and separated on a 8% SDS-PAGE. The proteins were then transferred to nitrocellulose membranes and after blocking, were probed with suitably diluted monoclonal and polyclonal antibodies. Antibodies used were HT-7 monoclonal antibody (BR-01, clone HT-7, Innogenetics) and Tau-5 monoclonal antibody (60101A, Pharmigen) both diluted 1:1000.

Results

Thy-1 Tau 40

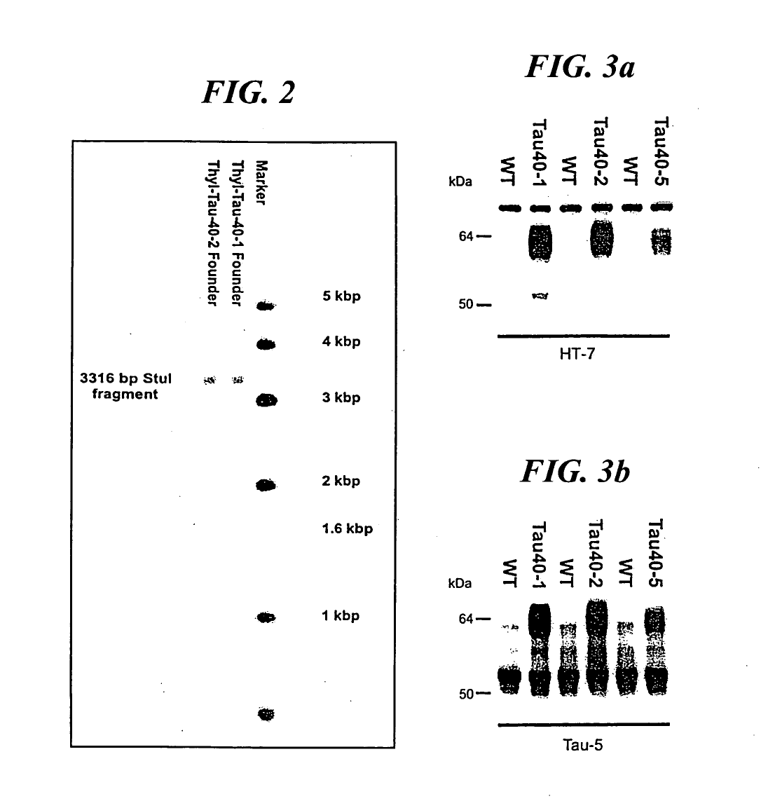

Expression of the human Tau 40 cDNA in the brain of transgenic mice was obtained using the Thy-1 promoter. Ten injection sessions yielded 46 pups (from 450 injected FVB oocytes transferred into 17 Fl pseudopregnant females). Genotyping by Southern blotting technique identified 5 out of the 46 pups as the human-Tau 40 founders (FIG. 2). Additional genotyping using duplex PCR identified these five mice as founders. The Fl offspring of the founder Thy-1 Tau 40/4 suffered from premature death and the complete strain died out within 2 months. Founder Thy-1 Tau 40/3 failed to reproduce normally and as a result three founder lines survived and appear “genetically” healthy. The Western results showed a ±70 kDa Tau protein in all the transgenic lines (FIG. 3) representing the longest human Tau isoform.

The transgene expression of all the founder lines reached comparable levels with this relation of Line 1 showing the highest expression, followed by Line 2 closely and then Line 5. The strain of Thy-1 Tau 40/5 is being bred into homozygous strains.

PAC2 Human Tau Gene Clone

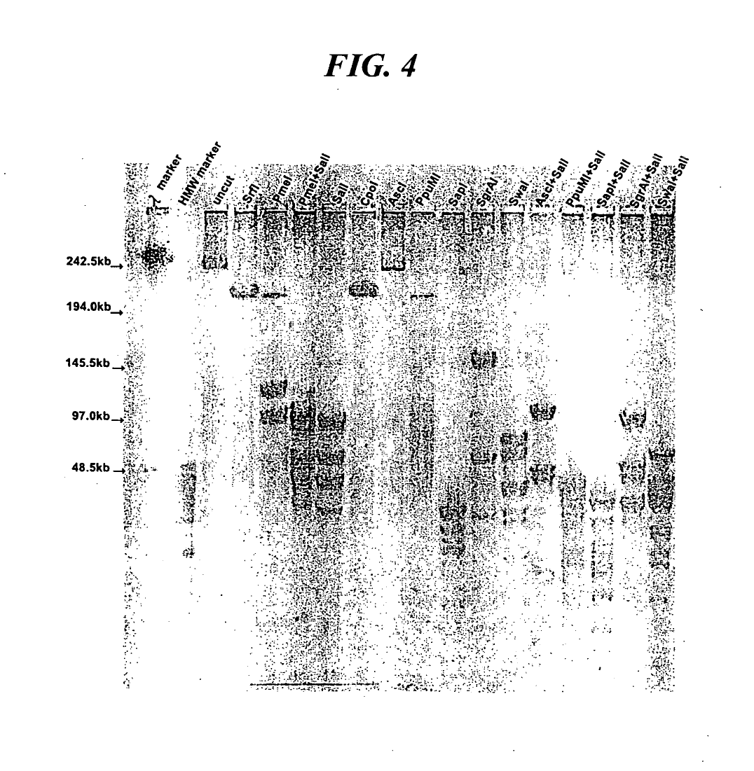

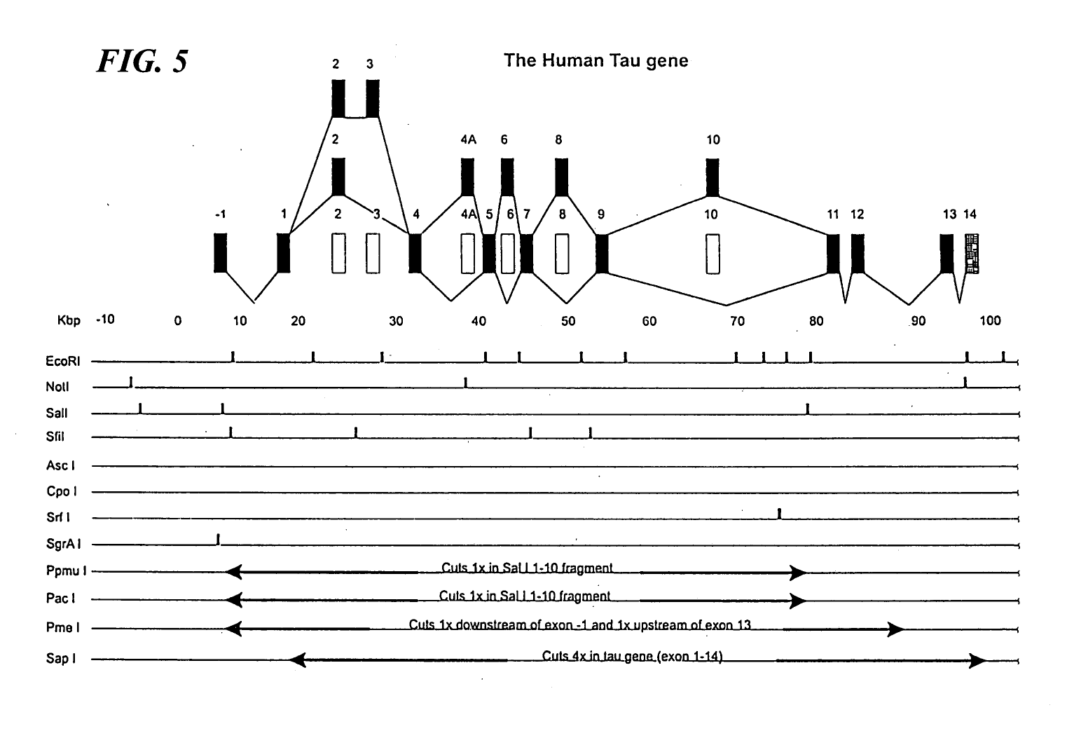

The purified PAC2 clone was characterised by the analyses of restriction fragments separated by Pulse Field Gel Electrophoresis (PFGE) and identified by Southern blotting using the probes generated by PCR (see materials and methods). The results of the different digestions using rare-cutting restriction enzymes are shown on the restriction map of the human Tau gene (FIG. 4). The PAC2 clone as sized by PFGE was around 200 kb and housed the entire human Tau gene, confirmed by Southern blotting using different probes that identified the 5′, middle and 3′ regions of the gene. To mention a few of the enzymes that were used to linearise the construct outside the Tau gene, we found that restriction with SalI and NotI enzymes gave 4 and 5 bands respectively, while Pmel cleaved the gene twice and Cpol linearised the clone (FIG. 5). The double digests of the PAC2 DNA of NotI and SalI with Cpol analysed on the PFGE demonstrated that one of the fragments made by the single digests of the NotI and Sall enzymes was cleaved but the Southern blotting done demonstrated that Cpo1 did not cleave any part of the human Tau gene. These analyses helped us to conclude that the PAC2 DNA could only be linearised by Cpol without fragmentation of the human Tau gene.

The linearised DNA was then purified using Qiagen columns and dialysis chambers [Millipore Purification columns, Spectra PorCE Dispodialyzer of Spectrum] of different pore sizes, of which we found the tip-20 column of QIAGEN the most efficient as it yielded DNA with least shearing and with a low elution volume a concentration of 1 ng/Fl was obtained, (one of the drawbacks of the dialyses membranes) which is required for microinjection. Genotyping by PCR identified 2 out of 9 pups as the PAC2 human Tau gene founders. Although the PCR did show us results yet no expression was observed in these mice as studies by Western Blotting.

Knockin-Knockout Targeted Vector

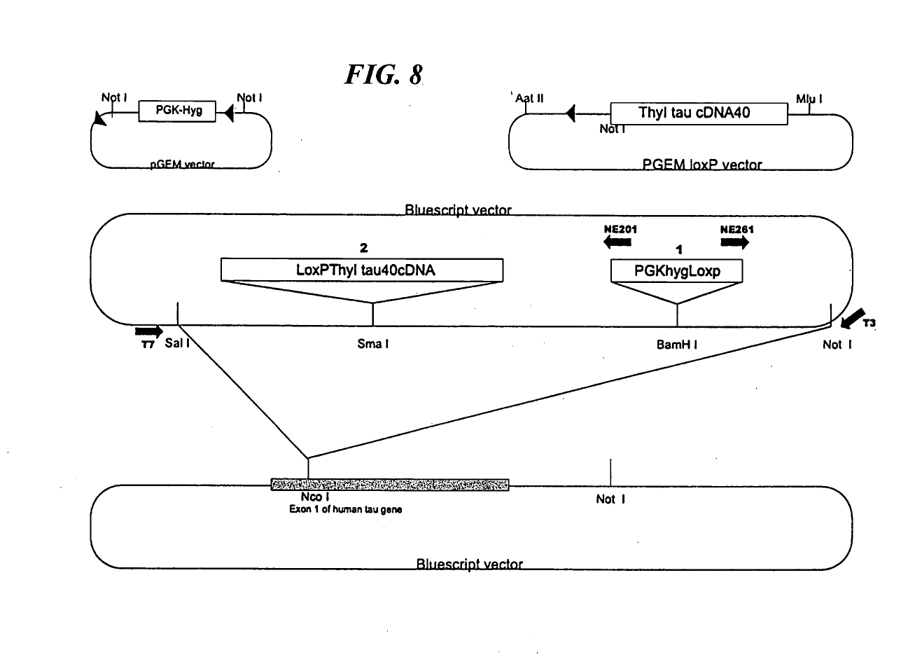

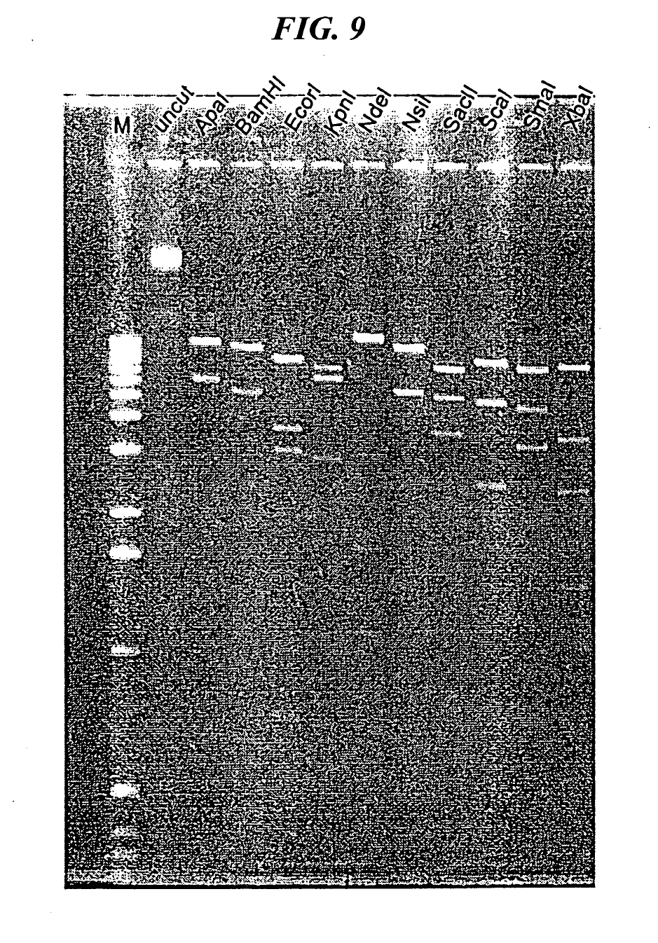

The loxP-PGK-hygromycin construct was cut out of the pGEM vector by NotI and ligated into the BamHI site of the Bluescript vector. Transformation of DH5α cells with this 4.7 kb construct yielded two positive colonies out of the 20 screened. Restriction analyses with SalI and ScaI enzymes gave the expected bands indicating that ligation occurred in the right orientation which was confirmed by sequencing with the T7 primer. The 8 kb Thy-1 human Tau 40 construct (see materials and methods) with the loxP site was subcloned into the SmaI site of the above bluescript vector. After transformation 5 out of the 20 colonies screened harboured the insert. Restriction analyses using ApaI, EcorV and Xmnl enzymes identified 2 colonies holding the Thy 1 Tau 40 insert in the desired orientation, additionally confirmed by sequencing with primers T7 and NE201 (sequence located in the PGK). The next step of the synthesis of the construct involved the introduction of the 10 kb Sal1-NotI fragment into the unique Ncol site of the exon 1 of the mouse Tau gene. 11 colonies were screened, of which 2 were found to contain the insert after transformation. Sequencing using the primers NE201 (5′-GATGTGGAATGTGTGCGA-3′ (SEQ ID NO:7)), NE260 (5′-CGCCAGGAGTTTGACA-3′ (SEQ ID NO:8)) (sequence located in the 5′ region of mouse exon 1) and NE261 (5′-CTCATTCCTCCCACTCAT-3′(SEQ ID NO:9)) (sequence located in the 5′ end of the PGK-hygromycin construct) was done to confirm the orientation expected and of the 2 colonies, only one had the right orientation. FIG. 8 gives an overall view of the making of the construct. Restriction analyses carried out using various enzymes as shown below indicated the presence of the complete construct in the right orientation (FIG. 9). The sizes have been estimated with the help of the 1 kb marker.

| | Band sizes as seen in the insert with the |
|---|---|
| Enzymes | right orientation |
| ApaI | ˜9.2 kb, ˜6.6 kb, ˜0.8 kb |
| BamHI | ˜0.2 kb, ˜0.4 kb, ˜0.7 kb, ˜0.9 kb, 4.9 kb, |
| | ˜9.5 kb |
| EcorI | ˜3.0 kb, ˜3.4 kb, 0.9 kb, ˜8.0 kb, 0.7 kb |
| KpnI | ˜6.5 kb, ˜7.0 kb, ˜2.9 kb |
| NdeI | ˜13.5 kb, ˜1.2 kb, ˜1.6 kb |
| NslI | ˜10.9 kb, ˜5.4 kb |
| SacII | ˜5.2 kb, ˜3.4 kb, ˜7.7 kb |
| ScaI | ˜8.7 kb, ˜2.5 kb, ˜5.2 kb |
| SmaI | ˜3.4 kb, ˜8.1 kb, ˜4.4 kb, 0.2 kb |
| XbaI | ˜8.7 kb, ˜2.4 kb, ˜3.4 kb, ˜0.6 kb, ˜1.2 kb |

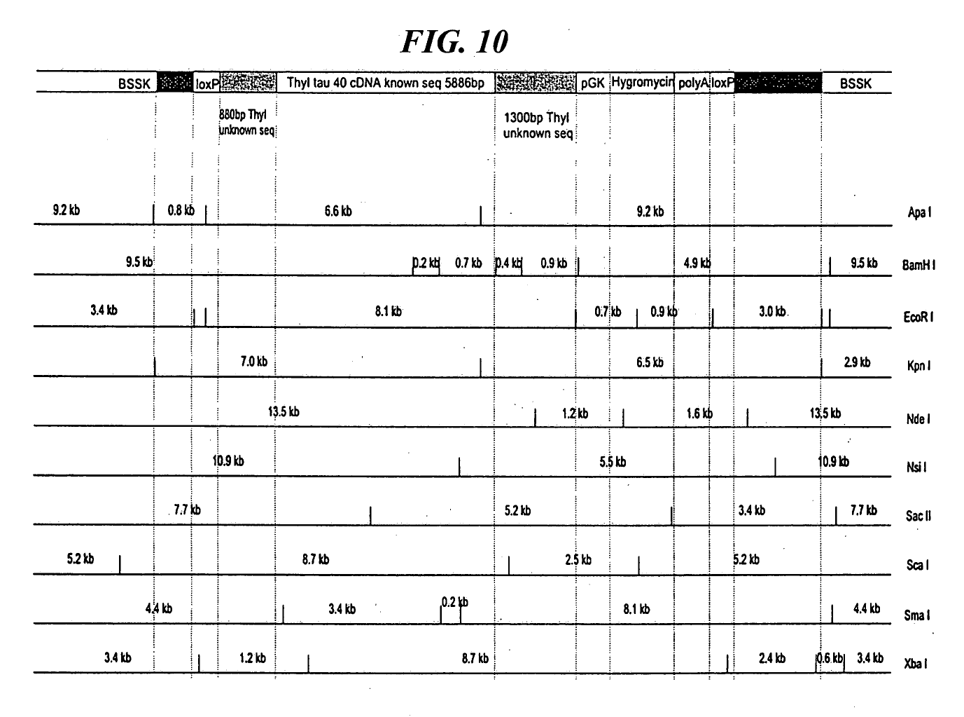

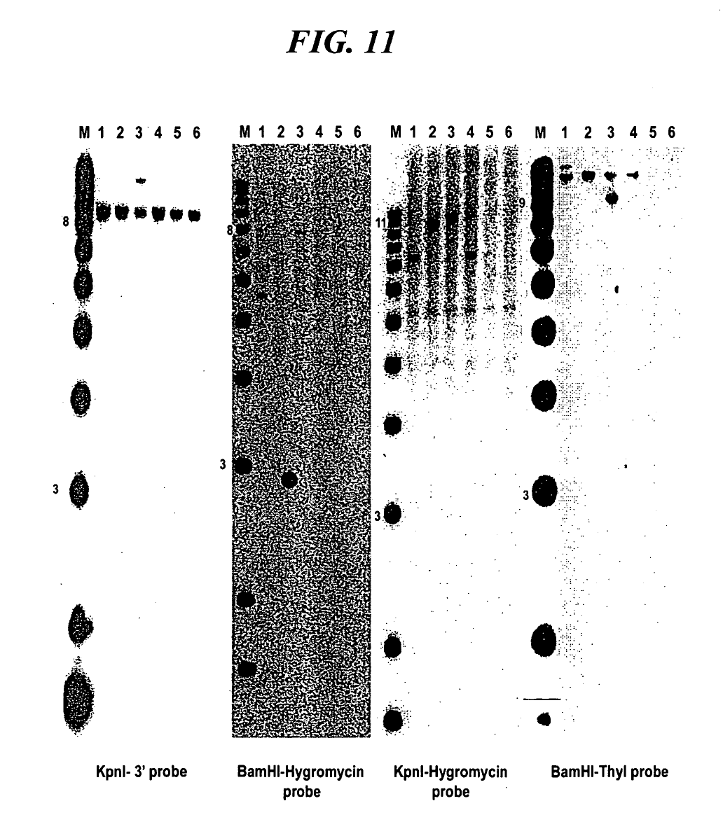

FIG. 10 shows a restriction map of the concluded construct. This construct is incapable of replicating and/or expressing the exogenous proteins in yeast. This final construct was linearised with NotI and purified on a tip-100 column (Qiagen) which finally gave a concentration of 2.25 Fg/Fl of which 8 Fl was used for electroporation into ES cells. The ES cells that survived the electroporation were grown on Hygromycin selective medium and after a fortnight well-grown 333 colonies had been picked up for culturing. With the help of Southern blotting using the external 3′ probe for the first screening (as mentioned in the materials and methods), we were able to pick up 6 potential positive cell lines in the first screening. After the second screening of these 6 colonies with the internal Hygromycin and ThyI Tau 40 probe we obtained one cell line that contained the right targeted construct in it. Besides, the ThyI probe used finally also confirmed the presence of the 5′ region of the construct in the positive cell line (FIG. 11) and the 5′ BamHI fragment hybridising with this probe measured the same number of base pairs as the predicted BamHI-fragment if the construct was homogonously recombined. The marker used in the blots is a 1 kb marker.

| | Digestion of | | Homologous | Type of |
|---|---|---|---|---|
| Probes | ES DNA | Band Size | Recombination | Probe |
| 3 = | KpnI | 11.2 kb | HR₁ | External probe |
| | 8.4 kb | No HR | | |
| Hygromycin | KpnI | 11.2 kb | HR | Internal probe |
| | BamHI | 7.9 kb | HR | Internal probe |
| ThyI Tau 40 | KpnI | 7.6 kb | | Internal probe |
| ThyI | BamHI | 8.9 kb | HR | Internal probe |

This first positive cell line was used for injection into blastocysts while further screening has resulted in five more potential cell lines. Uterine transfers have so far given 20 pups from three female mice of which 6 are chimeric.

### REFERENCES

Aberle H, Bauer A, Stappert J, Kispert A, Kemler R (1997) β-catenin is a target for the ubiquitin-proteasome pathway. EMBO J 16:13, 3797-3804.
Anderton B H, Brion J-P, Couck A-M, Davis D R, Gallo J-M, Hanger D P, Landhani K, Latimer D A, Lewis C, Lovestone S, Marquardt B, Miller C C J, Mulot S F C, Reynolds C H, Rubniak T, Smith C J, Woodgett J (1995) Modulation of PHF-like tau phosphorylation in cultured neurons and transfected cells. Neurobiology of Aging 16:3, 389-402.
Behrens J, Jerchow B-A, Wurtele M, Grimm J, Asbrand C, Wirtz R, Kühl M, Wedlich D, Birchmeier W (1998) Functional interaction of an axin homolog, conductin, with β-catenin, APC, and GSK-3β. Science 280:596-599.
Biernat J, Mandelkow E-M, Schröter C, Lichtenberg-Kraag B, Steiner B, Berling B, Meyer H, Mercken M, Vandermeeren A, Goedert M. and Mandelkow E (1992). The switch of tau protein to an Alzheimer-like state includes the phosphorylation of two serine-proline motifs upstream of the microtubule binding region. EMBO J. 11, 4:1593-1597.
Brion J-P, Hanger D P, Bruce M T, Couck A-M, Flament-Durant J, Anderton A (1991) Tau in Alzheimer neurofibriallary tangles. Biochem J 273:127-133.
Brownless J, Irving N G, Brion J-P, Gibb B J M, Wagner U, Woodgett J, Miller C C J (1997) Tau phosphorylation in transgenic mice expressing glycogen synthase kinase-3β transgenes. NeuroReport 8:3251-3255.
Goedert M, Jakes R, Crowther A, Cohen P, Vanmechelen E, Vandermeersen M, Cras P. (1994) Epitope mapping of monoclonal antibodies to the paired helical filaments of Alzheimer=s disease: identification of phosphorylation sites in tau protein. Biochem. J. 301, 871-877.
Geodert M., Spillantini, M. G., Jakes R., Rutherford, D. and Crowther, R. A. (1989) Multiple isoforms of human microtubule-associated protein tau: sequences and localization in neurofibrillary tangles of Alzheimer's disease. Neuron, 3, 519-526.
Götz J, Probst A, Spillantini M G, Schafer T. Jakes R, Burki K and Goedert M. (1995) Somatodendric localization and hyperphosphorylation of tau protein in transgenic mice expressing the longest human brain tau isoform. EMBO J. 14, 7, 1304-1313.
Guan R J, Khatra B S, Cohlberg J A (1991) Phosphorylation of bovine neurofilament proteins y protein kinase FA (glycogen synthase kinase 3). J Biol chem 266:8262-8267.
Hanger D P, Hughes K, Woodgett J R, Brion J-P, Anderton B H (1992) Glycogen synthase kinase-3 induces Alzheimer's disease-like phosphorylation of tau: generation of paired helical filament epitopes and neuronal localization of. the kinase. Neuroscience Letters 147:58-62.
Hasegawa M, Morishima-Kawashima M, Takoi K, Suzuki M, Titani K, Ihara Y (1992) Protein sequence and mass spectrometric analyses of tau in Alzheimer's disease brain. J Biol Chem 267:24, 17047-17054.
Ikeda S, Kishida S, Yamamoto H, Murai H, Koyama S, Kikuchi A (1998) Axin, a negative regulator of Wnt Signaling pathway, forms a complex with GSK-3β and β-catenin and promotes GSK-3β-dependent phosphorylation of β-catenin. EMBO J 17:5, 1371-1384.
Ishiguro K, Omori A, Takamatsu M, Sato K, Arioka M, Ushida T, Imahori K (1992a) Phosphorylation sites on tau by tau protein kinase I, a bovine derived kinase generating an epitope of paired helical filaments. Neuroscience Letters 148:202-206.
Ishiguro K, Shiratsuchi A, Sato S, Omori A, Arioka M, Kobayashi S, Uchida T, Imahori K (1993) Glycogen synthase kinase 3β is identical to tau protein kinase I generating several epitopes of paired helical filaments. FEBS 325:3, 167-172.
Ishiguro K, Takamatus M, Tomizawa K, Omori A, Takahashi M, Arioka M, Uchida T, Imahori K (1992b) Tau protein kinase I converts normal tau protein into A68-like component of paired helical filaments. J Biol Chem 267:15, 10897-10901.
Lovestone S, Reynolds C H (1997). The phosphorylation of tau: a critical stage in neurodevelopment end neurodegnerative processes. Neuroscience 78:2, 309-324.
Lovestone S, Hartley C L, Pearce J, Anderton B H (1996) Phosphorylation of tau by glycogen synthase kinase-3β intact mammalian cells: the effects on the organization and stability of microtubules. Neuroscience 73:4, 1145-1157.
Lovestone S, Reynolds C H, Latimer D, Davis D R, Anderton B H, Gallo J-M, Hanger D, Mulot S, Marquardt B, Stabel S, Woodgett J R, Miller C C J (1994) Alzheimer's disease-like phosphorylation of the microtubule-associated protein tau by glycogen synthase kinase-3 in transfected mammalian cells. Current Biology 4:1077-1086.
Mandelkow E M, Biernat J, drewes G, Gustke N, Trinczek B, Mandelkow E (1995) Tau domains, Phosphorylation, and interaction with microtubules. Neurobiology of aging 16, 3, 355-363.
Mandelkow E-M, Drewes G, Biernat J, Gustke N, Van Lint J, Vandenheede J R, Mandelkow, E (1992) Glycogen synthase kinase-3 and the Alzheimer-like state of microtubule-associated protein tau. FEBS 314:3, 315-321.
Moechars D, Lorent K, De Strooper B, Dewachter I, Van Leuven F (1996) Expression in brain of amyloid precursor protein mutated in the α-secretase site causes disturbed behaviour, neuronal degeneration and premature death in transgenic mice. EMBO J 15:6, 1265-1274.
Morris R (1992) Thy-1, the enigmatic extrovert on the neuronal surface. BioEssays 14:10, 715-722. Munoz-Montano, J. R., Morno, F. J., Avila J., Dias-Nido, J. (1997). Lithium inhibits Alzheimer's disease-like tau protein phosphorylation in neurons. FEBS Letters, 411, 183-188.
Otvos L, Feiner L, Lang E, Szendri G I, Goedert M and Lee V M-Y (1994). Monoclonal antibody PHF-1 recognizes tau protein phosphorylated at serine residues 396 and 404. J. Neurosci. Res. 39:669-673.
Pollanen M S, Markiewicz P, Goh M C (1997) Paired helical filaments are twisted ribbons composed of two parallel and aligned components: image reconstruction and modeling of filament structure using atomic force microscopy. J Neuropathol Exp Neurol 56:1, 79-85.
Preuss U, Biernat J, Mandelkow E M, Mandelkow E (1997) The Ajaws≈model of tau-microtubule interaction examined on CHO cells. J. Cell Science 110, 789-7800.
Sengupta A, Kabat J, Novak M, Wu Q, Grundke-Iqbal I, Iqbal K (1998) Phosphorylation of tau at both Thr231 and Ser262 is required for maximal inhibition of its binding to microtubules. Arch. Biochem. Biophys. 357, 2, 299-309.
Sperber B R, Leight S, Goedert M, Lee V M-Y (1995) Glycogen synthase kinase-3β phosphorylates tau protein at multiple sites in intact cells. Neuroscience Lett 197:149-153.
Stambolic V, and Woodgett J R (1994) Mitogen inactivation of glycogen synthase kinase-3β in intact cells via serine 9 phosphorylation. Biochem J 303:701-704.
Sutherland C, Leighton I A, Cohen P (1993) Inactivation of glycogen synthase Kinase-3β by phosphorylation: new kinase connections in insulin and growth-factor signalling. Biochem J 296:15-19.
Takahashi M, Tomizawa K, Kato R, Sato K, Uchida T, Fujita S C, Imahori K (1994) Localization and developmental changes of tau protein kinase I/glycogen synthase kinase-3β in rat brain. J Neurochem 63:245-255.
Tashiro K, Hasegawa M, Ihara Y and Iwatsubo T (1997) Somatodendric localization of phosphorylated tau in neonatal and adult rat cerebral cortex. NeuroReport 8: 2797-2801.
Trinczek B, Biernat J, Baumann K, Mandelkow E M, Mandelkow E (1995) Domains of tau proteins, differential phosphorylation, and dynamic instability of microtubules. Molec. Biol. Cell 6, 1887-1902.
Utton M A, Vandecandelaere A, Wagner U, Reynolds C H, Gibb G M, Miller C C C, Bayley P M, Anderton B H (1997) Phosphorylation of tau by glycogen synthase kinase 3β affects the ability of tau to promote microtubule self-assembly. Biochem J 323:741-747.
Van Lint J, Khandelwal R L, Merlevede W, Vandenheede J R (1993) A specific immunoprecipitation assay for the protein kinase FA/glycogen synthase kinase-3. Anal Biochem 208:132-137.
Wagner U, Utton M, Gallo J-M, Miller C C J (1996) Cellular phosphorylation of tau by GSK-3β influences tau binding to microtubules and microtubule organization. J Cell Science 109:1537- 1543.
Wischik, Novak M, Thøgersen H C, Edwards P C, Runswick M J, Jakes R, Walker J E, Milstein C, Roth M, Klug A (1988) isolation of a fragment of tau derived from the core of the paired helical filament of Alzheimer disease. Proc Natl Acad Sci USA 85:4506-4510.
Wood J G, Mirra S S, Pollock N J, Binder L I (1986) Neurofibrillary tangles of Alzheimer=s disease share antigenic determinants with the axonal microtubule-associated protein tau (tau). Proc Natl Acad Sci USA 83:4040-4043.
Yamaguchi H, Ishiguro K, Uchida T, Takashima A, Lemere C A, Imahori K (1996) Preferential labeling of Alzheimer neurofibrillary tangles with antisera for tau protein kinase (TPK) I/glycogen synthase kinase-3β and cyclin-dependent kinase 5, a component of TPK II. Acta Neuropathol 92:232-241.

1-51. (canceled)

52. A nucleic acid vector comprising:
(a) a nucleic acid sequence encoding a human Tau protein operably linked to
(b) a nucleic acid sequence that directs expression of said human Tau nucleic acid in the nervous system of a mouse;
(c) and a targeting sequence wherein said targeting sequence comprises a nucleotide sequence exhibiting a sufficient degree of homology with said sequence encoding said Tau protein to facilitate integration of said vector into the genome of said mouse by homologous recombination, so as to prevent expression of equivalent Tau protein or a related or equivalent protein from said mouse in favour of said human Tau protein.

53. The vector according to claim 52 further comprising a sequence encoding a reporter molecule.

54. The vector according to claim 53 wherein said reporter molecule comprises the hygromycin Pgk-hyg marker gene sequence.

55. The vector according to claim 52 wherein said sequence encoding human Tau is a cDNA sequence.

56. The vector according to claim 52 wherein the sequence directing expression is the Thy1 promoter.

57. The vector according to claim 52 wherein said targeting sequence comprises a NcoI restriction site corresponding to the unique NcoI restriction site of exon1 of the mouse wild type genome.

58. The vector according to claim 52 further comprising two loxP sites flanking either of the sequences of step (a) and (b).

59. The vector according to claim 52 further comprising a stop sequence capable of preventing expression of said human Tau protein and which sequence is flanked by two loxP sites capable of undergoing reciprocal conservative DNA recombination in the presence of Cre recombinase with the resulting excision of said stop sequence.

60. Isolated in vitro mouse cells transformed, transfected or injected with a vector according to claim 52.

61. The cells according to claim 60 which are embryonic cells.

67. The cells according to claim 61 wherein said embryonic cells are embryonic stem cells.
